(19) United States
(12) Patent Application Publication
(10) Pub. No.: US 2025/0099683 A1
(43) Pub. Date: Mar. 27, 2025

(54) Fluid Adhesive Dispenser Device for IV Port Securement or Wound Closure

(71) Applicant: Becton, Dickinson and Company, Franklin Lakes, NJ (US)

(72) Inventors: Rahul Malviya, Salmon Arm (CA); Shishir Prasad, Ramsey, NJ (US); Manish Kumar, Bengaluru (IN); Praveen Nalawade, Bangaluru (IN)

(21) Appl. No.: 18/729,601

(22) PCT Filed: Jan. 30, 2023
(86) PCT No.: PCT/US23/11870

§ 371 Date: Jul. 17, 2024

(30) Foreign Application Priority Data
Jan. 31, 2022 (IN) 202211005266

(51) Int. Cl. A61M 5/28; A61L 24/04

(52) U.S. Cl. CPC A61M 5/281; A61L 24/04

(57) ABSTRACT

A fluid adhesive dispenser device includes a housing having a passageway through the housing and a port at a distal end of the passageway. The dispenser device also includes a container at least partially within the housing configured to contain a single-use amount of the fluid adhesive. The container is configured to be opened allowing the fluid adhesive contained therein to be actively expelled from the container through the passageway and the port of the housing. A method of manufacture of the fluid adhesive dispenser device includes providing the dispenser device filled with the single-use amount of the fluid adhesive and sterilizing the filled device.

### CROSS-REFERENCE TO RELATED APPLICATION

The present application claims priority to Indian Provisional Application Ser. No. 20/221,1005266, entitled “Fluid Adhesive Dispenser Device for IV Port Securement or Wound Closure”, filed Jan. 31, 2022, the entire disclosure of which is hereby incorporated by reference in its' entirety.

### BACKGROUND OF THE INVENTION

### Field of the Invention

The present disclosure relates generally to fluid dispensing devices and, in particular, to a disposable and/or single-use fluid adhesive dispenser device for dispensing an amount of fluid adhesive to be used for medical procedures, such as intravenous (IV) port securement, insertion site sealing, and/or wound closure procedures.

### Description of Related Art

Fluid adhesives, such as curable polymeric solutions, are used for medical procedures, such as to secure an intravenous (IV) port to a patient's skin, as well as for wound closure and insertion site sealing to prevent or reduce microbial ingress. For example, minimally invasive surgery, such as laparoscopic surgery, is typically accomplished using small incisions. Such small incisions can be closed by traditional techniques, such as the introduction of sutures or staples to close the incision wounds. More recently, the use of surgical adhesives has gained favor in closing incisions and wounds.

Many different packaging configurations, dispenser devices, and applicators have been developed for providing a predetermined amount of the fluid adhesive to a specific surgical site, such as to a wound or insertion site. For example, fluid adhesives can be provided in disposable pouches or sleeves, which can be punctured or torn to release adhesive contained therein. Fluid adhesive can also be provided to a patient's skin using disposable tools, such as swabs, brushes, or sponges. More sophisticated dispenser and applicator devices can include sealed ampules, cartridges, vials, or other containers containing an amount of fluid adhesive to be provided to a wound or incision site. Many existing dispenser devices include a mechanism for opening the container and then allow the fluid adhesive to drain from the container by gravity. Other dispenser devices can include pumping or squeezing mechanisms for expelling the fluid adhesive from the dispenser device.

However, there is a need for improved dispenser devices that allow a user to precisely control fluid flow rate and volume to deposit a desired amount of the fluid adhesive at a desired surgical/wound site. The dispenser devices of the present disclosure are configured to provide such precise control of fluid release, while also providing long shelf-life and preventing contamination and leakage of the device and/or fluid adhesive contained therein prior to use.

### SUMMARY OF THE INVENTION

According to an aspect of the disclosure, a fluid adhesive dispenser device includes a housing having a passageway through the housing and a port at a distal end of the passageway. The dispenser device also includes a container at least partially within the housing configured to contain a single-use amount of the fluid adhesive. The container is configured to be opened, allowing the fluid adhesive contained therein to be actively expelled from the container through the passageway and the port of the housing.

According to another aspect of the disclosure, a method of manufacture of the previously described fluid adhesive dispenser device includes providing the dispenser device filled with the single-use amount of the fluid adhesive; and sterilizing the filled device.

According to another aspect of the disclosure, a fluid adhesive dispenser device includes a housing having a passageway through the housing and a port at a distal end of the passageway and an ampule at least partially within the housing. The ampule is configured to contain a single-use amount of the fluid adhesive and to be ruptured so that the fluid adhesive passes from an interior of the ampule to the passageway of the housing. The dispenser device also includes a filter in the passageway of the housing configured to separate broken pieces of the ruptured ampule from the fluid adhesive prior to expulsion of the fluid adhesive from the port of the housing.

According to another aspect of the disclosure, a fluid adhesive dispenser device configured to contain a single-use amount of a fluid adhesive includes a barrel having a proximal end, a distal end having a fluid port, and a sidewall extending between the proximal end and the distal end. The dispenser device also includes a pierceable proximal seal connected to an inner surface of the sidewall of the barrel and a pierceable distal seal connected to the inner surface of the sidewall of the barrel spaced apart from the proximal seal, such that the single-use amount of the fluid adhesive is contained within the barrel between the proximal seal and the distal seal. The dispenser device further includes a plunger disposed in and configured to move through the barrel to expel the fluid adhesive from the barrel through the fluid port. The plunger includes a piercing surface configured to pierce the proximal seal and/or the distal seal as the plunger moves through the barrel.

According to another aspect of the disclosure, a fluid adhesive dispenser device includes a housing having a reservoir portion configured to contain a single-use amount of a fluid adhesive, a passageway extending from the reservoir portion, and a port at a distal end of the passageway for expelling the fluid adhesive from the housing. The dispenser device also includes at least one pierceable seal in the passageway that can be pierced to permit flow of the fluid adhesive from the reservoir portion through the passageway and a removable cap attached to the port for preventing the fluid adhesive from being expelled through the port. The cap includes a spike configured to pierce the at least one seal when the spike is inserted into the passageway of the housing.

Non-limiting illustrative examples of embodiments of the present disclosure will now be described in the following numbered clauses.

Clause 1: A fluid adhesive dispenser device, comprising: a housing comprising a passageway through the housing and a port at a distal end of the passageway; and a container at least partially within the housing configured to contain a single-use amount of the fluid adhesive, wherein the container is configured to be opened, allowing the fluid adhesive contained therein to be actively expelled from the container through the passageway and the port of the housing.

Clause 2: The device of clause 1, wherein the fluid adhesive is expelled from the housing through the port of the housing in a direction-independent manner.

Clause 3: The device of clause 1 or clause 2, wherein the single-use amount of the fluid adhesive is less than about 1.0 mL, preferably less than about 0.5 mL, and more preferably less than about 0.2 mL.

Clause 4: The device of any of clauses 1-3, wherein the fluid adhesive is actively expelled from the container by reducing a fluid-containing volume of the container, thereby forcing fluid from the container into the passageway extending through the housing.

Clause 5: The device of any of clauses 1-4, wherein the fluid adhesive comprises a tissue adhesive configured to be used for securing a vascular access device to skin of a patient, wound closure, and/or insertion site closure.

Clause 6: The device of any of clauses 1-5, wherein the fluid adhesive comprises a cyanoacrylate composition.

Clause 7: The device of any of clauses 1-6, wherein the container is configured to be opened by at least one of compressing the container to create one or more openings in the container, rupturing the container, breaking the container, or piercing a portion of the container.

Clause 8: The device of any of clauses 1-7, wherein the housing comprises at least one compressible portion, such as a compressible bellows, configured to be compressed by a user to compress the container in order to expel the fluid adhesive from the container.

Clause 9: The device of any of clauses 1-8, wherein an inner surface of the housing is ultrasonically bonded to an outer surface of the container.

Clause 10: The device of any of clauses 1-9, wherein the housing comprises a base comprising a cavity with the container in the cavity and a cover connected to the base extending over the cavity, and wherein the cover is flexible and configured to be pressed to rupture the container and expel the fluid adhesive from the container into the passageway of the housing.

Clause 11: The device of any of clauses 1-10, wherein the container comprises glass and/or plastic configured to rupture when a compressing force is manually applied to the container by a user.

Clause 12: The device of any of clauses 1-11, wherein the housing comprises a pouch comprising a cavity sized to receive the container.

Clause 13: The device of clause 12, wherein the pouch comprises a metal coated polymer film.

Clause 14: The device of clause 12 or clause 13, wherein the container comprises a sealed ampule or bulb positioned in the cavity of the pouch, and wherein the fluid adhesive is sealed in the container without air.

Clause 15: The device of any of clauses 12-14, wherein the port comprises a nozzle attached to the pouch in fluid communication with the cavity of the pouch, such that the fluid adhesive is expelled from the cavity of the pouch through the nozzle as the pouch is compressed.

Clause 16: The device of clause 15, wherein the pouch is heat sealed to the nozzle, thereby forming a leak proof joint between the pouch and the nozzle.

Clause 17: The device of any of clauses 12-16, further comprising a notched seal extending across the passageway of the housing restricting fluid flow from the pouch through the port of the housing, wherein applying a force to the pouch breaks the notched seal so that the fluid adhesive can flow from the interior of the pouch to the port.

Clause 18: The device of any of clauses 1-17, wherein the port comprises an elongate tubular nozzle configured to be manually compressed by the user to control fluid flow through the nozzle.

Clause 19: The device of any of clauses 1-18, wherein the port is configured such that the fluid adhesive is expelled from the port as drops

Clause 20: The device of any of clauses 1-19, wherein the housing comprising at least one tab configured to contact an outer surface of the container to rupture the container.

Clause 21: The device of clause 20, wherein applying force to the at least one tab of the housing in a first direction ruptures the container, establishing fluid communication between an interior of the container and the passageway of the housing, and applying force to the housing in a second direction moves the container towards the port of the housing, thereby expelling the fluid adhesive from the interior of the container through the passageway and port of the housing.

Clause 22: The device of clause 21, wherein the housing further comprises a grip, such as a flange, and wherein applying force in the second direction comprises applying force to the grip to move the port of the housing towards the container.

Clause 23: The device of any of clauses 1-22, wherein the container comprises at least one pierceable seal that can be pierced to establish fluid communication between an interior of the container and the passageway of the housing.

Clause 24: The device of any of clauses 1-23, further comprising a filter within the passageway of the housing configured to separate broken pieces of the container from the fluid adhesive prior to expulsion of the fluid adhesive from the port of the housing.

Clause 25: The device of clause 24, wherein the filter comprises a porous member with openings sized to filter glass chards and/or plastic particles from the tissue adhesive.

Clause 26: The device of any of clauses 1-25, wherein the housing comprises a barrel comprising a proximal end, a distal end comprising the port, and a sidewall extending between the proximal end and the distal end.

Clause 27: The device of clause 26, wherein the barrel comprises a rigid thermoplastic material, such as polyester, polycarbonate, polypropylene, polyethylene, polyethylene terephthalate, or acrylonitrile butadiene styrene.

Clause 28: The device of clause 26 or clause 27, further comprising a plunger configured to move through the barrel to expel the fluid adhesive from the container through the passageway of the housing.

Clause 29: The device of clause 28, further comprising a plunger rod connected to the plunger for moving the plunger through the barrel.

Clause 30: The device of clause 29, wherein the plunger rod comprises a thumb press plate at a proximal end, and wherein the barrel comprises at least one flap for grasping the barrel to press the plunger rod into the barrel.

Clause 31: The device of any of clauses 28-30, wherein the plunger comprises one or more annular ribs configured to contact an inner surface of the sidewall.

Clause 32: The device of any of clauses 28-31, wherein the container comprises proximal and distal pierceable seals connected to an inner surface of the sidewall of the barrel, and wherein the fluid adhesive is contained between the first and second seals.

Clause 33: The device of clause 32, wherein the proximal and/or distal seal comprises a metal foil, such as aluminum foil.

Clause 34: The device of clause 33, wherein the plunger comprises a piercing surface. such as a spike, configured to pierce the proximal and/or distal seals.

Clause 35: The device of clause 34, wherein advancing the plunger through the barrel pierces the proximal seal and forces the fluid adhesive through the barrel and through the port.

Clause 36: The device of any of clauses 28-35, wherein movement of the plunger through the barrel causes the container to rupture, thereby allowing the fluid adhesive to move through the barrel and port.

Clause 37: The device of clause 36, wherein the container comprises a sealed bulb formed from a flexible material, the device further comprising a protective cover, preferably comprising coated aluminum film, at least partially surrounding the bulb, positioned between the plunger and the bulb.

Clause 38: The device of clause 37, wherein the port of the housing comprises a tubular nozzle, partially received within the distal end of the barrel, and wherein the plunger pushes the bulb against a proximal surface of the tubular nozzle to rupture the bulb.

Clause 39: The device of any of clauses 26-38, wherein the container comprises a proximal pierceable seal over an opening at the proximal end of the barrel, and a distal seal over the port.

Clause 40: The device of clause 39, further comprising a cap comprising a spike on the proximal end of the barrel, wherein the cap is configured to be compressed, such that the spike contacts and pierces the proximal seal.

Clause 41: The device of clause 40, wherein, once the proximal seal is pierced, repeatedly compressing the cap pushes the fluid adhesive through the barrel, thereby expelling the adhesive through the port.

Clause 42: The device of any of clauses 39-41, wherein the distal seal is configured to be removed by the user.

Clause 43: The device of any of clauses 1-42, further comprising a removable cap attached to the port for preventing the fluid adhesive from being expelled through the port until the cap is removed.

Clause 44: The device of clause 43, wherein the cap is removed from the port by twisting the cap relative to the port.

Clause 45: The device of clause 43 or clause 44, wherein the passageway of the housing comprises a compressible tubular nozzle configured to be compressed to control flow of the fluid adhesive through the port of the housing.

Clause 46: The device of clause 45, wherein the cap comprises an annular sidewall that at least partially encloses the tubular nozzle of the housing supporting the tubular nozzle preventing the tubular nozzle from being compressed until the cap is removed.

Clause 47: The device of any of clauses 43-46, wherein the container comprises a pierceable seal for retaining the adhesive in the container, and wherein the cap comprises a spike configured to pierce the seal when the spike is inserted into the passageway of the housing.

Clause 48: The device of clause 47, wherein the cap is initially provided in a first orientation with the spike pointing away from the port of the housing, and wherein the cap is configured to be removed from the port and reconnected to the port in a second orientation with the spike inserted into the port to pierce the seal.

Clause 49: A method of manufacture of the fluid adhesive dispenser device of any of clauses 1-48, the method comprising: providing the dispenser device of any of clauses 1-48, filled with the single-use amount of the fluid adhesive; and sterilizing the filled device.

Clause 50: The method of clause 49, wherein providing the dispenser device comprises: filling the container with the single-use amount of the adhesive; and positioning the filled container in the housing.

Clause 51: The method of clause 50, further comprising separately sterilizing the filled container prior to positioning the container in the housing.

Clause 52: The method of any of clauses 49-51, wherein filling the container comprises filling the container with from about 0.05 mL to about 1.0 mL of the fluid adhesive.

Clause 53: The method of any of clauses 49-52, wherein the fluid adhesive comprises a tissue adhesive configured to be used for securing a vascular access device to skin of a patient, wound closure, and/or insertion site closure.

Clause 54: The method of any of clauses 49-53, further comprising inserting a removable cap over the port of the housing.

Clause 55: The method of any of clauses 49-54, further comprising inserting a plunger into an open proximal end of the housing, wherein the plunger is configured to move through the housing to expel the fluid adhesive from the housing.

Clause 56: The method of any of clauses 49-55, wherein sterilizing the filled device comprises exposing the filled device to radiation.

Clause 57: The method of any of clauses 49-56, wherein, following sterilization, the tissue adhesive remains substantially unpolymerized for at least about 24 months.

Clause 58: A fluid adhesive dispenser device, comprising: a housing comprising a passageway through the housing and a port at a distal end of the passageway; an ampule at least partially within the housing configured to contain a single-use amount of the fluid adhesive and configured to be ruptured so that the fluid adhesive passes from an interior of the ampule to the passageway of the housing; and a filter within the passageway of the housing configured to separate broken pieces of the ruptured ampule from the fluid adhesive prior to expulsion of the fluid adhesive from the port of the housing.

Clause 59: The device of clause 58, wherein an inner surface of the housing is ultrasonically bonded to an outer surface of the container.

Clause 60: The device of clause 58 or clause 59, wherein the housing comprises a compressible bellows configured to be compressed by a user to compress the container in order to expel the fluid adhesive from the container.

Clause 61: The device of clause 60, wherein the housing further comprising at least one tab configured to contact an outer surface of the ampule to rupture the ampule.

Clause 62: The device of clause 61, wherein applying an inwardly directed force to the at least one tab of the housing moves the housing toward the ampule to rupture the ampule, and applying a distally directed force to the housing compresses the bellows, thereby moving the ampule towards the port of the housing to expel the fluid adhesive from an interior of the ampule through the passageway and port of the housing.

Clause 63: The device of any of clauses 58-62, wherein the ampule comprises plastic and/or glass.

Clause 64: The device of clause 63, wherein the filter comprises a porous member with openings sized to filter glass chards and/or plastic particles from the tissue adhesive.

Clause 65: A fluid adhesive dispenser device configured to contain a single-use amount of a fluid adhesive, the device comprising: a barrel comprising a proximal end, a distal end comprising a fluid port, and a sidewall extending between the proximal end and the distal end; a pierceable proximal seal connected to an inner surface of the sidewall of the barrel; a pierceable distal seal connected to the inner surface of the sidewall of the barrel spaced apart from the proximal seal, such that the single-use amount of the fluid adhesive is contained within the barrel between the proximal seal and the distal seal; and a plunger disposed in and configured to move through the barrel to expel the fluid adhesive from the barrel through the fluid port, the plunger comprising a piercing surface configured to pierce the proximal seal and/or the distal seal as the plunger moves through the barrel.

Clause 66: The device of clause 65, wherein the barrel comprises a rigid thermoplastic material, such as polyester, polycarbonate, polypropylene, polyethylene, polyethylene terephthalate, or acrylonitrile butadiene styrene.

Clause 67: The device of clause 65 or clause 66, wherein the barrel contains a volume of fluid adhesive of less than about 1.0 mL, preferably less than about 0.5 mL, and more preferably less than about 0.2 mL, between the proximal seal and the distal seal.

Clause 68: The device of any of clauses 65-67, further comprising a plunger rod connected to the plunger for moving the plunger through the barrel.

Clause 69: The device of clause 68, wherein the plunger rod comprises a thumb press plate at a proximal end, and wherein the barrel comprises at least one flap for grasping the barrel to press the plunger rod into the barrel.

Clause 70: The device of any of clauses 65-69, wherein the plunger comprises one or more annular ribs configured to contact an inner surface of the sidewall.

Clause 71: The device of any of clauses 65-70, wherein the proximal and/or distal seal comprises a metal foil, such as aluminum foil.

Clause 72: The device of any of clauses 65-71, wherein advancing the plunger through the barrel pierces the proximal seal and forces the fluid adhesive through the barrel and through the fluid port

Clause 73: A fluid adhesive dispenser device, comprising: a housing comprising a reservoir portion configured to contain a single-use amount of a fluid adhesive, a passageway extending from the reservoir portion, and a port at a distal end of the passageway for expelling the fluid adhesive from the housing; at least one pierceable seal in the passageway that can be pierced to permit flow of the fluid adhesive from the reservoir portion through the passageway; and a removable cap attached to the port for preventing the fluid adhesive from being expelled through the port, the cap comprising a spike configured to pierce the at least one seal when the spike is inserted into the passageway of the housing.

Clause 74: The device of clause 73, wherein the cap is initially provided in a first orientation with the spike pointing away from the port of the housing, and wherein the cap is configured to be removed from the port and reconnected to the port in a second orientation with the spike inserted into the port to pierce the at least one seal.

Clause 75: The device of clause 74, further comprising removable packaging over the spike when the cap is in the first orientation that is removed to expose the spike prior to moving the cap to the second orientation.

Clause 76: The device of any of clauses 73-75, wherein the passageway of the housing comprises a compressible tubular nozzle configured to be compressed to control flow of the fluid adhesive through the port of the housing.

Clause 77: The device of clause 76, wherein the cap comprises an annular wall that at least partially encloses the tubular nozzle of the housing supporting the tubular nozzle and preventing the tubular portion from being compressed until the cap is removed.

Clause 78: The device of clause 76 or clause 77, wherein a channel extending through the compressible tubular nozzle is sized so that the fluid adhesive is expelled from the port of the housing as drops.

Clause 79: The device of any of clauses 73-78, wherein the single-use amount of the fluid adhesive is less than about 1.0 mL, preferably less than about 0.5 mL, and more preferably less than about 0.2 mL.

Clause 80: The device of any of clauses 73-79, wherein the reservoir portion of the housing is compressible, configured to be manually compressed by the user to force the fluid adhesive from the reservoir portion into the passageway of the housing.

Clause 81: The device of any of clauses 73-80, wherein the at least one seal comprises a metal foil, such as an aluminum foil.

### BRIEF DESCRIPTION OF THE DRAWINGS

FIG. 1A is a side view of a fluid adhesive dispenser device, according to an aspect of the present disclosure.

FIG. 1B is a cross-sectional view of the dispenser device of FIG. 1A.

FIG. 2A is a perspective view of another example of a fluid adhesive dispenser device, according to an aspect of the present disclosure.

FIG. 2B is a cross-sectional view of the dispenser device of FIG. 2A.

FIG. 3A is a perspective view of another example of a fluid adhesive dispenser device, according to an aspect of the present disclosure.

FIG. 3B is an exploded perspective view of the dispenser device of FIG. 3A.

FIG. 3C is a cross-sectional view of the dispenser device of FIG. 3A.

FIG. 4A is a perspective view of another example of a fluid adhesive dispenser device, according to an aspect of the present disclosure.

FIG. 4B is a cross-sectional view of the dispenser device of FIG. 4A.

FIG. 5A is a perspective view of another example of a fluid adhesive dispenser device, according to an aspect of the present disclosure.

FIG. 5B is another perspective view of the dispenser device of FIG. 5A, with a transparent or see-through housing.

FIG. 6A is a perspective view of another example of a fluid adhesive dispenser device. according to an aspect of the present disclosure.

FIG. 6B is an exploded perspective view of the dispenser device of FIG. 6A.

FIG. 6C is a cross-sectional view of the dispenser device of FIG. 6A;

FIG. 6D is a cross-sectional view of the dispenser device of FIG. 6A showing a plunger of the device in a final position.

FIG. 7A is a perspective view of another example of a fluid adhesive dispenser device, according to an aspect of the present disclosure.

FIG. 7B is an exploded perspective view of the dispenser device of FIG. 7A.

FIG. 7C is a cross-sectional view of the dispenser device of FIG. 7A.

FIG. 8A is a perspective view of another example of a fluid adhesive dispenser device, according to an aspect of the present disclosure.

FIG. 8B is an exploded perspective view of the dispenser device of FIG. 8A.

FIG. 8C is a cross-sectional view of the dispenser device of FIG. 8A.

FIG. 8D is an enlarged cross-sectional view of the dispenser device of FIG. 8A.

FIG. 8E is another cross-sectional view of the dispenser device of FIG. 8A, showing a spike on a removable cap of the device inserted into a passageway of the device.

FIG. 8F is a drawing showing a hand of a user pressing on an elongated tubular portion of the dispenser device of FIG. 8A to control flow of the fluid adhesive from the device.

FIG. 9 is a side view of another example of a fluid adhesive dispenser device, according to an aspect of the present disclosure.

FIG. 10 is a flow chart showing a method for manufacturing a fluid adhesive dispenser device, according to an aspect of the present disclosure.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. As used herein, the term “proximal” refers to a portion or end of a device, such as a fluid delivery device or syringe, configured to be grasped, manipulated, or used by a practitioner, clinician, or another user. The term “distal” refers to an end or portion of the device that is farthest away from the portion of the device that is grasped, manipulated, or used by the practitioner. For example, the “proximal end” of a syringe refers to the end including a plunger rod and grip. The “distal end” of the syringe refers to the end with a nozzle or needle cannula for expulsion of fluid from the syringe. However, it is to be understood that the invention may assume alternative variations and step sequences, except where expressly specified to the contrary. It is also to be understood that the specific devices and processes illustrated in the attached drawings, and described in the following specification, are simply exemplary embodiments of the invention. Hence, specific dimensions and other physical characteristics related to the embodiments disclosed herein are not to be considered as limiting.

With reference to the figures, the present disclosure is directed to fluid adhesive applicator or dispenser devices 10, 110, 210, which can be configured to deliver a single-use amount of a fluid adhesive F to a target site. As used herein, a “fluid adhesive” refers to an adhesive composition that remains in a fluid (e.g., flowable) state for an extended period of time (e.g., up to or longer than 24 months) when contained within the dispenser device 10, 110, 210. For example, the fluid adhesive F may be a liquid, solution, or gel when contained in the dispenser device 10, 110, 210 at room temperature. After being expelled from the dispenser device 10, 110, 210, the fluid adhesive F may dry, harden, or cure to, for example, attach objects together or seal an opening or incision.

In some examples, the fluid adhesive F is a tissue adhesive, also referred to as surgical glue, skin glue, or liquid stitches, configured to adhere to skin of a patient. The tissue adhesive can be used, for example, for securing a vascular access device, such as an intravenous (IV) port, to the patient's skin. The tissue adhesive can also be used for wound closure or insertion site sealing to prevent microbial ingress. The dispenser devices 10, 110, 210 of the present disclosure can be filled with a variety of different types of biocompatible tissue adhesives, as are known in the art. For example, the tissue adhesive contained in the dispenser device 10, 110, 210 can be a cyanoacrylate composition.

In some examples, the dispenser devices 10, 110, 210 of the present disclosure are configured to be single-use and/or disposable devices configured to be disposed of after delivering a single dose or single-use amount of the fluid adhesive F. As used herein, a “single-use amount” of the fluid adhesive F refers to an amount of adhesive needed for performing common medical actions including, but not limited to, IV port securement, wound closure for small wounds or incisions, and/or insertion site sealing. For example, the dispenser device 10, 110, 210 can be configured to contain less than about 1.0 mL, preferably less than 0.5 mL, or more preferably less than about 0.2 mL of the fluid adhesive F. In other examples, the dispenser device 10, 110, 210 can contain from about 0.05 mL to about 1.0 mL of the fluid adhesive F.

In some examples, the fluid adhesive F is contained within a sealed container 12, 112, 212, such as an ampule, fluid reservoir, sealed barrel, bulb, pouch, or sachet, positioned within or enclosed by other components of the dispenser device 10, 110, 210. Desirably, the sealed container 12, 112, 212 is a low volume enclosure including minimal air or dead space so that the dispenser device 10, 110, 210 can be as small as possible. Further, the dispenser device 10, 110, 210 is configured to expel all or nearly all of the fluid adhesive F from the container 12, 112, 212, meaning that the container 12, 112, 212 does not need to contain excess or extra adhesive to account for an amount of adhesive that remains in (i.e., cannot be expelled from) the device 10, 110, 210 after use.

In some examples, the dispenser device 10, 110, 210 is configured to open the sealed container 12, 112, 212 to release the single-use amount of the fluid adhesive F contained therein and then expel the released fluid adhesive F from the device 10, 110, 210. For example, when the container 12, 112, 212 is formed from a material that easily cracks or shatters, opening the container 12, 112, 212 can include applying sufficient force to the container 12, 112, 212 to crack or shatter the material. In other examples, opening the container 12, 112, 212 can include compressing the container 12, 112, 212 to increase a fluid pressure within the container 12, 112, 212 until the container 12, 112, 212 ruptures or bursts, thereby creating one or more openings in the container 12, 112, 212 for the fluid adhesive F to flow through. Opening the container 12, 112, 212 can also include piercing a portion of the container 12, 112, 212, such as a pierceable septum or seal of the container 12, 112, 212, to release the fluid adhesive F.

Once the container 12, 112, 212 is opened, the dispenser devices 10, 110, 210 of the present disclosure are configured to actively expel the fluid adhesive F from the container 12, 112, 212 and device 10, 110, 210. As used herein, “active” expulsion of the fluid adhesive F from the container 12, 112, 212 means that some portion or component of the device 10, 110, 210 is operated or actuated to force the fluid adhesive F from the container 12, 112, 212 and device 10, 110, 210. For example, the fluid adhesive F can be actively expelled from the container 12, 112, 212 by operating the device 10, 110, 210 to reduce a fluid-containing volume of the container 12, 112, 212, thereby forcing the fluid adhesive F from the container 12, 112, 212 into another portion of the device 10, 110, 210 and then from the device 10, 110, 210 through, for example, a fluid port or nozzle of the device 10, 110, 210.

By contrast, “passive” expulsion of fluid refers to movement of fluid by environmental or inherent forces, such as gravity, natural elasticity of components of the container or device, osmosis, differential pressure, etc. Due to the active expulsion of the fluid adhesive F from the container 12, 112, 212 and device 10, 110, 210, the dispenser devices 10, 110, 210 of the present disclosure are direction-independent or can be operated in a direction-independent manner. As used herein, “direction-independent” operation means that the dispenser device 10, 110, 210 can be used in any orientation or positioned (e.g., right-side-up, inverted, sideways, etc.) while expelling the fluid adhesive F from the device 10, 110, 210. Further, the dispenser devices 10, 110, 210 of the present disclosure can be activated (e.g., the container 12, 112, 212 can be opened) and used to dispense the fluid adhesive F from the device 10, 110, 210 with the device 10, 110, 210 in the same orientation. By contrast, a device that expels fluid by gravity or other environmental forces may only operate in an expected manner in a specific orientation. Further, in some cases, conventional dispenser devices may need to be held in one orientation to activate the device and then inverted so that the fluid contained in the device can drain from the device by gravity. Due to the “direction-independent” operation of the dispenser devices 10, 110, 210, users can release the fluid adhesive F with substantial precision and control to locations that would be difficult to access if the device 10, 110, 210 needed to be held in a specific orientation in order to activate the device 10, 110, 210 and/or to release the fluid adhesive F from the device 10, 110, 210. The active expulsion and direction-independent operation of the dispenser devices 10, 110, 210 of the present disclosure also allow for precise control of flow rate as the fluid adhesive F is expelled from the device 10, 110, 210.

### Fluid Adhesive Dispenser Devices with Breakable Container and Filter

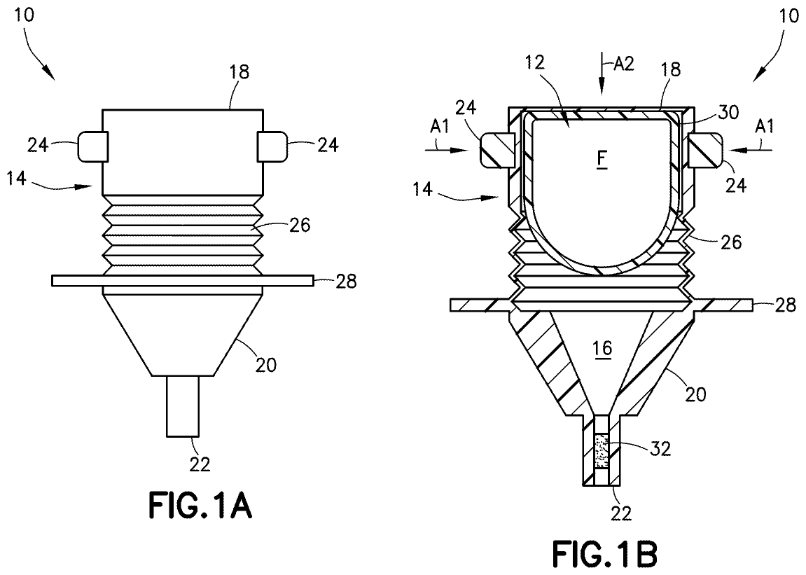

With reference to FIGS. 1A and 1B, an exemplary fluid adhesive dispenser device 10 comprises a housing 14 that includes or defines a passageway 16 and a container 12 at least partially within the housing 14. For example, the housing 12 can be a hollow structure with a bulbous proximal portion 18 sized to receive the container 12 and a narrow distal portion 20 comprising a fluid port 22 or nozzle for expelling the fluid adhesive F from the device 10. The housing 14 can be formed from a flexible or bendable material that can be pressed by a user, such as a medical practitioner, clinician, or another trained or untrained caregiver, to apply force to the container 12. For example, the housing 14 can be formed from a thermoplastic material including portions that are sufficiently thin to move, bend, or flex when forces are applied to the housing 14 by the user. Exemplary thermoplastic materials that can be used to form portions of the housing can include polyester, polycarbonate, polypropylene, polyethylene, polyethylene terephthalate, acrylonitrile butadiene styrene, or other injection moldable or formable resin materials, as are known in the art. The container 12 can be fixed to and/or secured within the housing 14 by conventional manufacturing processes, as are known in the art. For example, an outer surface of the container 12 can be secured to an inner surface of the housing 14 by ultrasonic welding, heat sealing, or using conventional adhesives or fasteners.

In some example, the housing 14 further includes portions configured to be pressed by a user to apply force to the container 12. For example, as shown in FIGS. 1A and 1B, the housing 14 comprises opposing activator tabs 24 extending radially outwardly from the proximal portion 18 of the housing 14. The activator tabs 24 can be configured to apply an inwardly directed force to the housing 14, causing the housing 14 to contact and apply pressure to the container 12. The housing 14 can also include an axially compressible bellows 26 positioned between the proximal portion 18 and the distal portion 20 of the housing 14. The bellows 26 can be configured such that applying a distally directed force to the housing 14 compresses the bellows 26, thereby moving the container 12 towards the distal portion 20 of the housing 14 to expel the fluid adhesive F from an interior of the container 12 into the passageway 16 of the housing 14. The housing 14 can also include a grip or support, such as a radially extending flange 28, which allows a user to manipulate the dispenser device 10 with one hand. For example, the user can grasp the dispenser device 10 between fingers and thumb, with the fingers supporting the flange 28 and the thumb pressing against the proximal portion 18 of the housing 14 to compress the bellows 26.

In some examples, the container 12 is a sealed ampule 30 formed from a breakable material, such as glass or rigid plastic, configured to break, rupture, or shatter when sufficient force is applied to the ampule. As shown in FIG. 1B, the ampule 30 can be a gumdrop shape (i.e., having a flat proximal surface and a narrow dome-shaped distal surface) secured in the proximal portion 18 of the housing 14. The ampule 30 can be configured to contain the single-use amount of the fluid adhesive F, such as less than about 1.0 mL of the fluid adhesive F.

The dispenser device 10 further comprises a filter 32 within the nozzle 22 or passageway 16 of the housing 14 configured to separate broken pieces of the ruptured ampule 30 from the fluid adhesive F prior to expulsion of the fluid adhesive F from the port of the housing 14. For example, the filter 32 can be a porous sheet or elongated structure, such as a cylinder, with openings sized to filter glass chards and/or plastic particles from the fluid adhesive F.

The dispenser device 10 can initially be provided to the user filled with the single-use amount of the fluid adhesive F and contained within protective packaging (not shown), such as blister packaging. The user initially removes the dispenser device 10 from the packaging. The user then presses the activator tabs 24 radially inwards (shown by arrows A1 in FIG. 1B), causing the inner surface of the housing 14 to press against the ampule 30. When sufficient pressure is applied to the ampule 30, the ampule 30 ruptures or bursts causing the fluid adhesive F in the ampule 30 to flow from an interior of the ampule 30 to the passageway 16 of the housing 14. After the ampule 30 bursts, the user can grasp the device 10 with, for example, a thumb on the proximal portion 18 of the housing 14 and fingers below (i.e., distal to) the flange 28. Applying pressure to the proximal portion 18 of the housing 14 (as shown by arrow A2 in FIG. 1B) and flange 28 causes the bellows 26 of the housing 14 to compress moving the ampule 30 through the housing 14 in a distal direction, thereby forcing the fluid adhesive F from the ampule 30 into the passageway 16. Continuing to apply pressure to the proximal portion 18 of the housing 14 and flange 28 forces the fluid adhesive F through the filter 32, where any glass charts or plastic particles of the ruptured ampule 30 are filtered. The filtered fluid adhesive F is then expelled from the device 10. As previously described, the fluid adhesive F can be expelled to a patient's skin for IV port securement, wound closure, insertion site sealing, and similar uses.

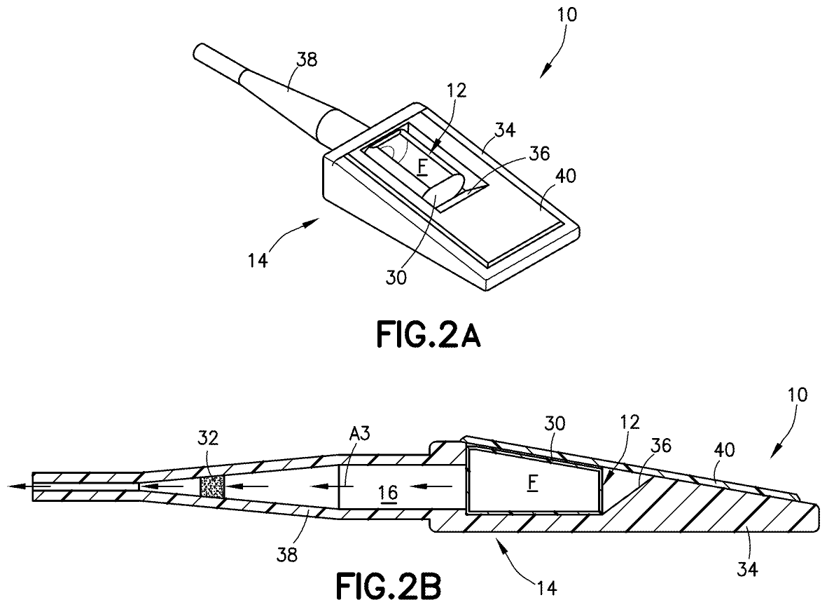

FIGS. 2A and 2B show another exemplary fluid adhesive dispenser device 10. As in previous examples, the dispenser device 10 comprises a housing 14 that includes a passageway 16 and a port or nozzle 22 at a distal portion 20 or end of the housing 14 for expelling the fluid adhesive F from the device 10. The dispenser device 10 also includes the container 12 within the housing 14 configured to contain the fluid adhesive F and the filter 32, positioned in the passageway 16, fluid port, or nozzle 22 of the housing 14 for removing portions of the ruptured container 12 from the fluid adhesive F.

As shown in FIGS. 2A and 2B, the housing 14 includes a base portion 34, which defines a cavity 36 sized to receive the container 12, and a tubular portion 38 extending from the base portion 34 to the nozzle 22 of the housing 14. The base portion 34 and the tubular portion 38 of the housing 14 can be formed from an opaque or translucent rigid material, such as a molded thermoplastic material. The housing 14 also includes a cover 40 connected to the base portion 34 and extending over the cavity 36. For example, the cover 40 can be connected to the base portion 34 and/or to a top surface of the container 12 by ultrasonic welding. The cover 40 can be a transparent or translucent sheet formed from a flexible material. The cover 40 can be configured to be pressed by the user to compress the container 12, causing the container 12 to rupture or break to release the fluid adhesive contained in the container 12 into the cavity 36 of the housing 14.

As shown in FIGS. 2A and 2B, the container 12 is a sealed ampule 30 that is generally rectangular in shape embedded in a rectangular cavity 36. As in previous examples, the sealed ampule 30 can be formed from a material that breaks or shatters when sufficient force is applied to the ampule 30. The dispenser device 10 of FIGS. 2A and 2B operates in a similar manner to the previous examples. Specifically, when ready for use, the user presses on the cover 40 to break or rupture the ampule 30. Once the ampule 30 breaks or ruptures, the user continues to apply force to the ampule 30 to expel the adhesive from the ampule into the passageway 16 extending through the tubular portion 38 of the housing 14. For example, the user may slide a finger over the surface of the cover 40 to push the fluid adhesive F from a proximal end of the ampule 30 towards a distal end of the ampule 30. Continuing to apply force to the cover 40 causes the fluid adhesive F to pass through the passageway 16 (in a direction of arrow A3 in FIG. 2B), through the filter 32 to remove chards of glass and/or plastic particles, and through the nozzle 22 at the distal end 20 of the housing 14.

### Fluid Adhesive Dispenser Devices with Flexible Pouches or Sachets

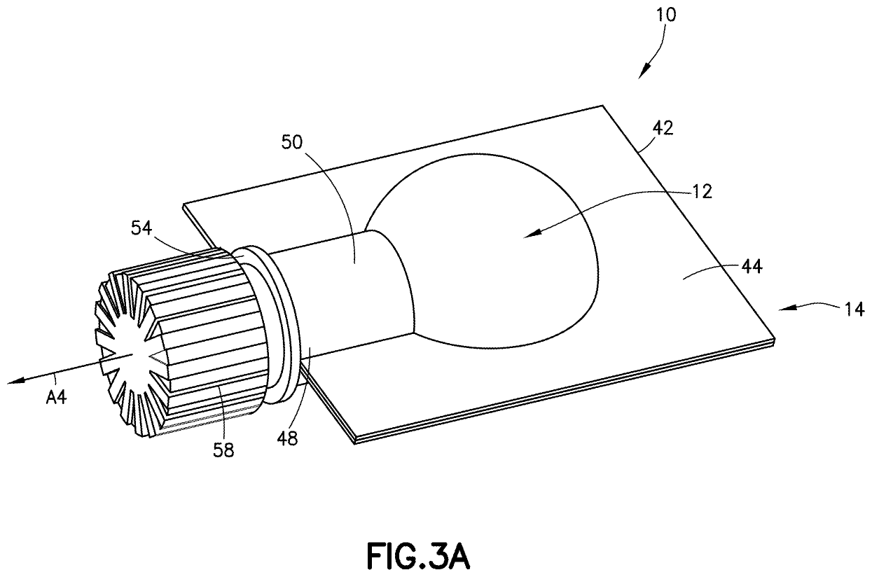

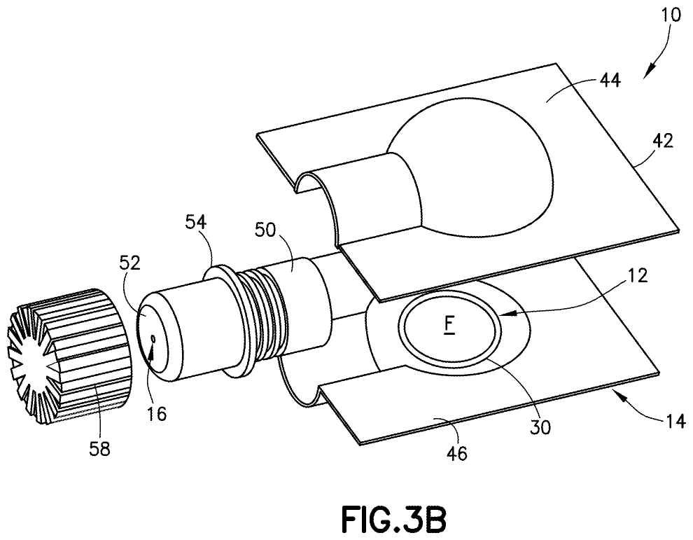

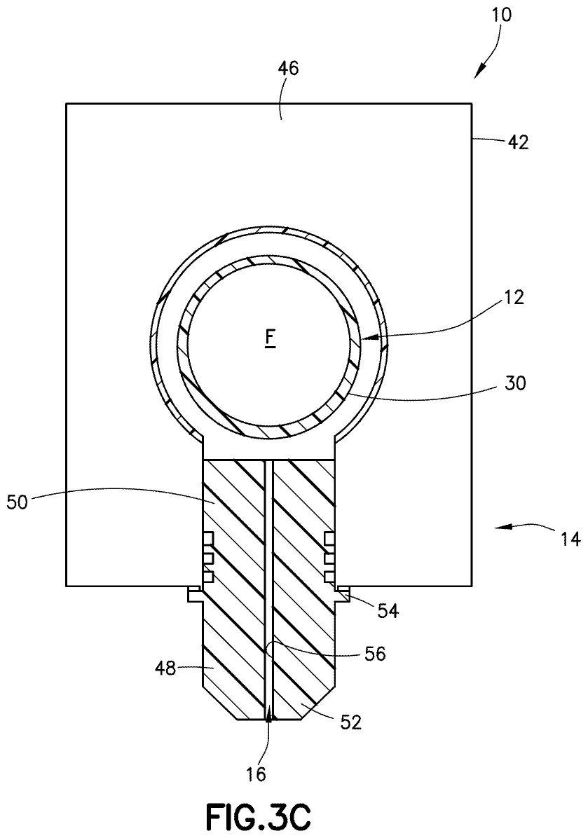

With reference to FIGS. 3A-3C, another exemplary fluid adhesive dispenser device 10 includes housing 14 comprising a sealed pouch 42 that defines a bulge or cavity 36 sized to receive a container 12, such as a sealed ampule 30 or bulb, containing the fluid adhesive F, and a passageway 16 extending from the cavity 36 towards a peripheral edge of the pouch 42. The pouch 42 can include an upper or first sheet 44 and a second or lower sheet 46 of a flexible material, such as a metal foil or a polymer film, such as a metal-coated polymer film, that are joined together to enclose the sealed ampule 30.

As previously described, the sealed ampule 30 or bulb contains the single-use amount of the fluid adhesive F. The ampule 30 can be formed from a breakable material, such as glass or plastic. As in previous examples, the ampule 30 can be configured to break when force is applied to the dispenser device 10 by a user. A bulb can be formed from a flexible material. The bulb can be configured to compress as force is applied to the bulb, eventually bursting or rupturing to release the fluid adhesive F contained in the bulb. The bulb or ampule 30 can be spherical or cylindrical in shape and sized to fit in the cavity 36 or bulge of the pouch 42. In other examples, the bulge or cavity 36 of the pouch 42 can be directly filled with the fluid adhesive F without a separate container or enclosure. In that case, the pouch 42 may include one or more breakable seals (not shown in FIGS. 3A-3C) in the passageway 16 of the pouch 42 for restricting the fluid adhesive F from moving through the passageway 16 until a user is ready to dispense the fluid adhesive F from the pouch 42.

The dispenser device 10 also includes a nozzle 48 or tip inserted into the pathway 16 of the pouch 42 for expelling the fluid adhesive F from the device 10. As shown in FIGS. 3A-3C, the nozzle 48 or tip is a tubular structure comprising a proximal portion 50, a distal portion 52, and a central flange 54 between the proximal portion 50 and the distal portion 52. The proximal portion 50 of the nozzle 48 is inserted into the pouch 42 and can include protrusions, such as annular ridges, threads, or sealing joints, configured to retain the proximal portion 50 of the nozzle 48 in the pouch 42. In some examples, the nozzle 48 and sheets 44, 46 of the pouch 42 can be connected together by heat sealing to prevent the nozzle 48 from being removed from the pouch 42 and to prevent the sheets 44, 46 from being separated. As shown in FIG. 3C, the nozzle 48 also includes a narrow central lumen 56 extending through the proximal portion 50 and the distal portion 52 of the nozzle 48 for expulsion of the fluid adhesive F from the device 10. In some examples, the lumen 56 is sized or configured so that the fluid adhesive F is expelled from the lumen 56 as individual drops. For example, the lumen 56 may be tapered and/or may have a minimum diameter that is sized to prevent a steady stream of the fluid adhesive F from passing through the nozzle 48. Instead, due to friction of the fluid adhesive F against an inner surface of the nozzle 48, only individual drops are expelled from the nozzle 48.

In some examples, the nozzle 48 can include a filter (not shown in FIGS. 3A-3C), similar to filters of previous examples, positioned in the lumen 56. As in previous examples, the filter can be a sheet or another porous structure with openings sized so that the fluid adhesive F passes through the filter while debris, such as chards of glass or plastic particles from the ampule 30 or bulb, are retained by the filter. In other examples, the nozzle 48 can be sufficiently narrow to prevent unwanted debris, such as the glass chards or plastic particles, from passing through the nozzle 48, while allowing all or substantially all of the fluid adhesive F in the ampule 30 to pass through the nozzle 48 and to be dispensed from the device 10.

In some examples, the dispenser device 10 also includes a removable cap 58 configured to be positioned over the distal portion 52 of the nozzle 48. For example, the removable cap 58 can slide over the nozzle 48 with an open end of the cap 58 positioned proximate to the flange 54 of the nozzle 48. The cap 58 is positioned to protect the nozzle 48 and to prevent fluids or other debris from passing into the nozzle 48 or pouch 42 contaminating the device 10. The cap 58 can also prevent the fluid adhesive F from flowing out of the nozzle 48 until the user is ready to dispense the fluid adhesive F from the device 10. In some examples, the distal portion 52 of the nozzle 48 can include connectors, such as threads, configured to engage corresponding structures on an inner surface of the cap 58. In other examples, the cap 58 and nozzle 48 can include other common engagements or connections, such as a luer connection, snap fit connection, or interference connection, as are known in the art, for securing the cap 58 to the nozzle 48.

The dispenser device 10 can initially be provided to a user with the removable cap 58 over the distal portion 52 of the nozzle 48, as shown in FIG. 3A. Advantageously, no further packaging may be needed for the device 10, because the cap 58 effectively seals the lumen 56 of the nozzle 48 preventing contamination of the device 10 or premature expulsion of the fluid adhesive F from the device 10. Alternatively, the dispenser device 10 can be provided in disposable packaging that can be removed by the user before use of the dispenser device 10.

To use the dispenser device 10, the user first presses on the bulge or cavity 36 of the pouch 42 to rupture the ampule 30, thereby releasing the fluid adhesive F from the ampule 30 into the cavity 36 and/or passageway 16 of the pouch 42. When ready to dispense the fluid adhesive F, the user removes the cap 58 from the device 10 by, for example, twisting the cap 58 relative to the nozzle 48 and/or pulling the cap 58 away from the nozzle 48 (as shown by arrow A4 in FIG. 3A) with sufficient force to overcome any engagement between the nozzle 48 and cap 58. The user then presses on the bulge or cavity 36 of the pouch 42 to move the fluid adhesive F through the pouch 42 and into the lumen 56 of the nozzle 48. The fluid adhesive F then passes through the filter (if present) in the lumen 56 of the nozzle 48 to remove any debris, such as glass chards or plastic particles, from the fluid adhesive F. Continuing to apply pressure to the pouch 42 expels the fluid adhesive F from the distal portion 52 of the nozzle 48. As previously described, in some examples, the nozzle 48 is configured so that the fluid adhesive F is released as drops, rather than as a constant stream of fluid adhesive F.

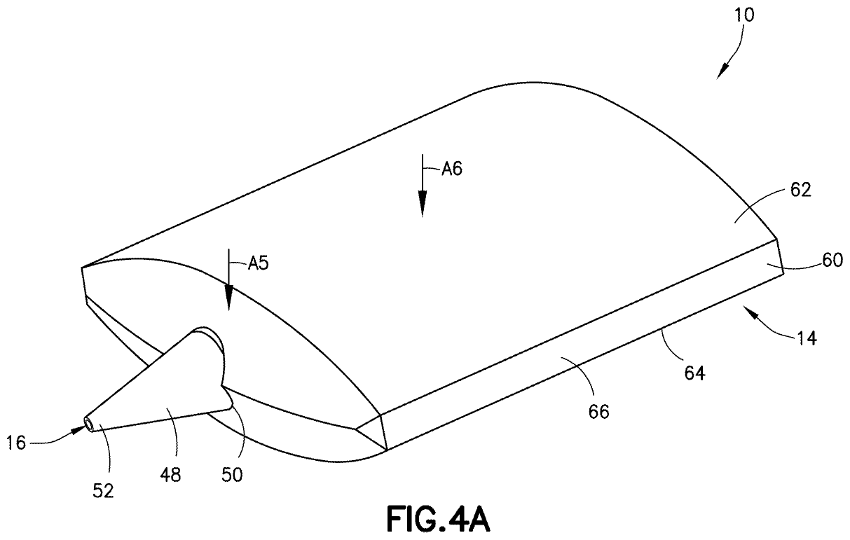

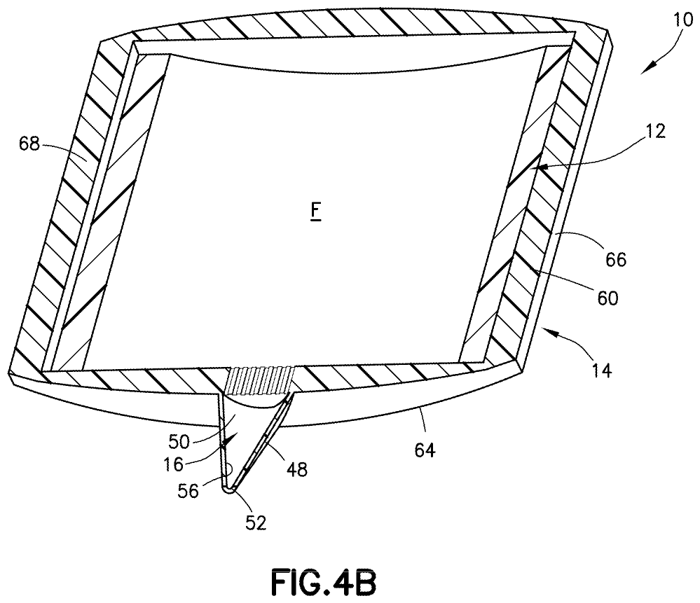

FIGS. 4A and 4B show another exemplary fluid adhesive dispenser device 10 including a pouch 42 or sachet and a conical nozzle 48 having an open proximal end portion 50 inserted into the pouch 42 and a narrowed distal end portion 52 external to the pouch 42. The pouch 42 of FIGS. 4A and 4B is formed from a multi-layer housing or packaging comprising an outer housing or packaging 60 formed from a flexible film or substrate. For example, the film or substrate can be a metal foil or a polymer film, such as a metal-coated polymer film. As shown in FIGS. 4A and 4B, the outer housing or packaging 60 is substantially rectangular in shape having a top surface 62, a bottom surface 64, and a peripheral edge surface 66 extending between the top surface 62 and the bottom surface 64.

An inner surface of the outer housing or packaging 60 is lined with or mounted to an inner packaging 68 formed from a film or substrate. The inner packaging 68 forms a container 12 suitable for containing a single-use amount of the fluid adhesive F. For example, the inner packaging 68 can be connected to the outer housing or packaging 60 by lamination, adhesives, heat sealing, or other mounting processes, as are known in the art. The inner packaging 68 can be formed from foil or film layers including, for example, a metal coated polymer film. The inner packaging 68 is a similar shape to the outer housing or packaging 60 and is sized to fit tightly within the outer housing or packaging 60 with minimal empty space between layers of the inner and outer packaging 60, 68. In other examples, the dispenser device 10 can instead include an ampule or bulb (not shown in FIGS. 4A and 4B) enclosed within the outer housing or packaging 60. As in previous examples, the ampule or bulb can contain the single-use amount of the fluid adhesive F. The user can rupture the ampule or bulb to release the fluid adhesive F into the interior of the pouch 42 by applying sufficient force to the outer housing or packaging 60 to break or rupture the ampule or bulb.

The dispenser device 10 further comprises the conical nozzle 48, which can be mounted through the peripheral edge surface 66 of the outer housing or packaging 60. As in previous examples, the conical nozzle 48 can be sealed to the pouch 42 by heat sealing to form a liquid-tight engagement between components of the dispenser device 10. As shown in FIG. 4B, in some examples, the outer packaging 60 and/or inner packaging 68 form a seal between the nozzle 48 and the interior of the pouch 42 containing the fluid adhesive F. For example, portions of the outer and/or inner packaging 60, 68 can extend over the open proximal end portion 50 of the nozzle 48 preventing the fluid adhesive F from passing into the nozzle 48 until a user is ready to dispense the fluid adhesive F from the dispenser device 10. In some examples, the portion of the outer packaging 60 and/or inner packaging 68 over the open proximal end portion 50 of the nozzle 48 can include a notch, crack, or perforation to facilitate and reduce a force needed to break the seal to establish fluid communication between the interior of the pouch 42 and the nozzle 48. The notch, crack, or perforation can be configured to open when sufficient force is applied to the pouch 42, thereby allowing the fluid adhesive F to pass into the nozzle 48 to be expelled from the dispenser device 10.

In order to use the dispenser device 10, the user first removes any external packaging from the device 10. The user then presses or pinches a portion of the pouch 42 over the seal between the proximal end portion 50 of the nozzle 48 and the interior of the pouch 42, as shown by arrows A5 (in FIG. 4A), to crack or open the seal. Once the seal is opened, the user applies pressure to a central portion of the pouch 42, as shown by arrows A6 (in FIG. 4A), in order to squeeze the fluid adhesive F through the opened seal and into the nozzle 48. In the nozzle 48, the fluid adhesive F can pass through a filter (if present) to remove plastic particles or other debris from the fluid adhesive F. The fluid adhesive F is then expelled from the device 10 through the distal end portion 52 of the nozzle 48.

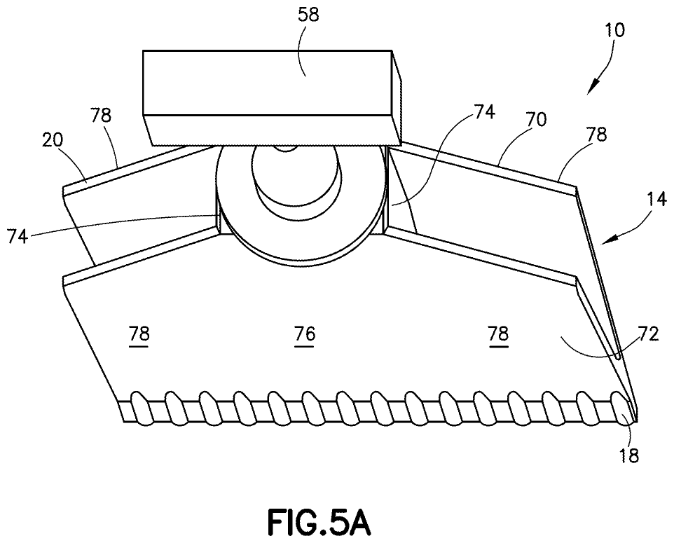

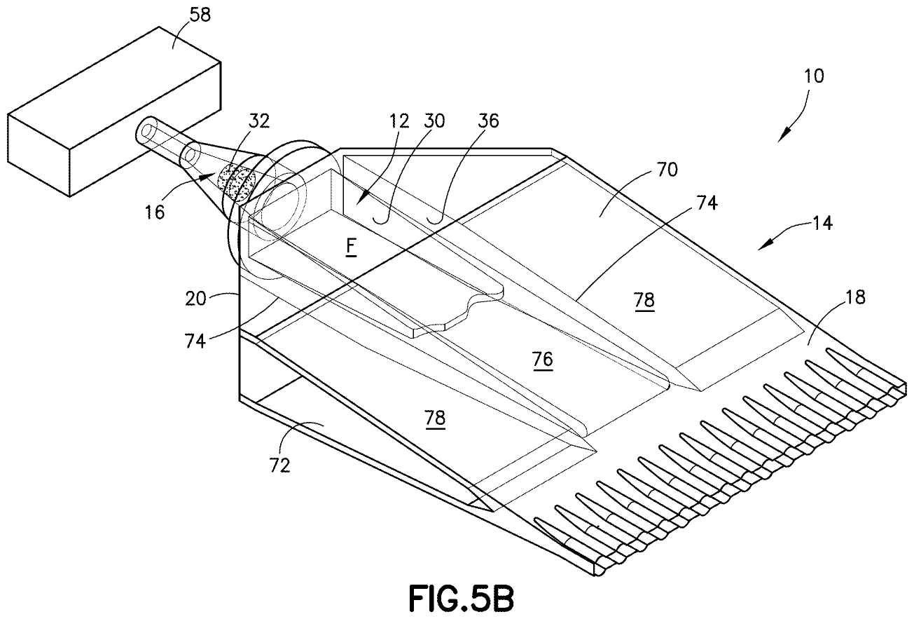

FIGS. 5A and 5B show another example of a fluid adhesive delivery device 10 including a sealed container 12 of fluid adhesive enclosed within an external housing 14 formed from a flexible and/or compressible material. The dispenser device 10 of FIGS. 5A and 5B differs from previous examples in the shape and configuration of the housing 14. Specifically, the housing 14 of FIGS. 5A and 5B is configured to be easier to compress or squeeze than previous examples due to the shape and configuration of portions of the housing 14.

As shown in FIGS. 5A and 5B, the dispenser device 10 includes a housing 14 comprising a first or upper flat sheet 70 and a second or lower flat sheet 72 connected together by connecting sheets 74 extending between the upper and lower sheets 70, 72. The upper and lower sheets 70, 72, as well as the connecting sheets 74, can be formed from flexible and/or bendable materials, such as molded or extruded thermoplastic materials. In some examples, the sheets 70, 72, 74 can be formed from multiple layers that are laminated together. The sheets 70, 72, 74 can be connected together by heat sealing, ultrasonic welding, or other suitable joining methods, as are known in the art. The housing 14 has a generally tapered shape that is narrower near a proximal end portion 18 of the housing 14 and wider near the distal end portion 20 of the housing 14. In order to provide the tapered shape, the connecting sheets 74 are generally triangular in shape, with longer sides connected to the upper sheet 70 and the lower sheet 72, a short side proximate to the distal end portion 20 of the housing 14, and a point proximate to the proximal end portion 18 of the housing 14.

As shown in FIG. 5B, the device 10 also includes a central cavity 36 or interior enclosed by central portions 76 of the upper and lower sheets 70, 72 and the connecting sheets 74 that is sized to contain the adhesive-filled container. The upper and lower sheets 70, 72 also include flap portions 78 on either side of the central portion 76, which extend beyond the cavity 36 or interior of the housing 14.

The dispenser device 10 of FIGS. 5A and 5B also includes a nozzle 48 connected to the distal end portion 20 of the housing 14. The nozzle 48 includes a proximal end portion 50 that is connected to the container 12. The nozzle 48 also includes a distal end portion 52 extending away from the container 12 for expelling the fluid adhesive F from the device 10. As in previous examples, the nozzle 48 can be a conical or frusto-conical shape, having a wider open proximal end portion 50 and a narrow distal end portion 52. A portion of the container 12 can extend over or between the open proximal end portion 50 of the nozzle 48 and the interior of the container 12 forming a seal, which prevent the fluid adhesive F in the container 12 from flowing into the nozzle 48 until a user is ready to dispense the fluid adhesive F from the device. As in previous examples, once the seal is opened or ruptured the fluid adhesive F flows from the container 12 into the nozzle 48.

As shown in FIGS. 5A and 5B, the device 10 can also include a removable cap 58 over the distal end portion 52 of the nozzle 48. As in previous examples, the removable cap 58 can be provided to prevent fluids or other debris from entering the nozzle 48 or device 10, which may contaminate the device 10. The removable cap 58 can also prevent the fluid adhesive F from being expelled from the nozzle 48 until the user is ready to dispense the fluid adhesive F from the device 10.

In use, the user first presses or pinches portions of the upper and/or lower sheets 70, 72 of the housing 14 over the seal between the nozzle 48 and the container 12. The applied force breaks or opens the seal between the interior of the container 12 and the nozzle 48, thereby establishing fluid communication between the interior of the container 12 and the nozzle 48. When the user is ready to dispense the fluid adhesive from the device 10, the user removes the cap 58 from the distal end portion 52 of the nozzle 48 by, for example, twisting the cap 58 relative to the nozzle 48 and/or pulling the cap 58 away from the nozzle 48. The user then slides a finger over the central portion 76 of the upper and/or lower sheets 70, 72 from the proximal end portion 18 of the housing 14 towards the distal end portion 20 of the housing 14 to squeeze the fluid adhesive F from the interior of the container 12 into the nozzle 48. Continuing to apply pressure to the container 12 pushes the fluid adhesive F through the nozzle 48 and expels the fluid adhesive F from the device 10 through the distal end portion 52 of the nozzle 48.

### Fluid Adhesive Dispenser Devices with Plungers

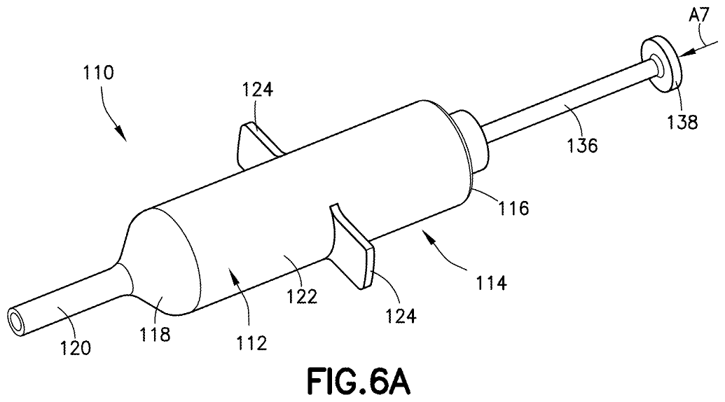

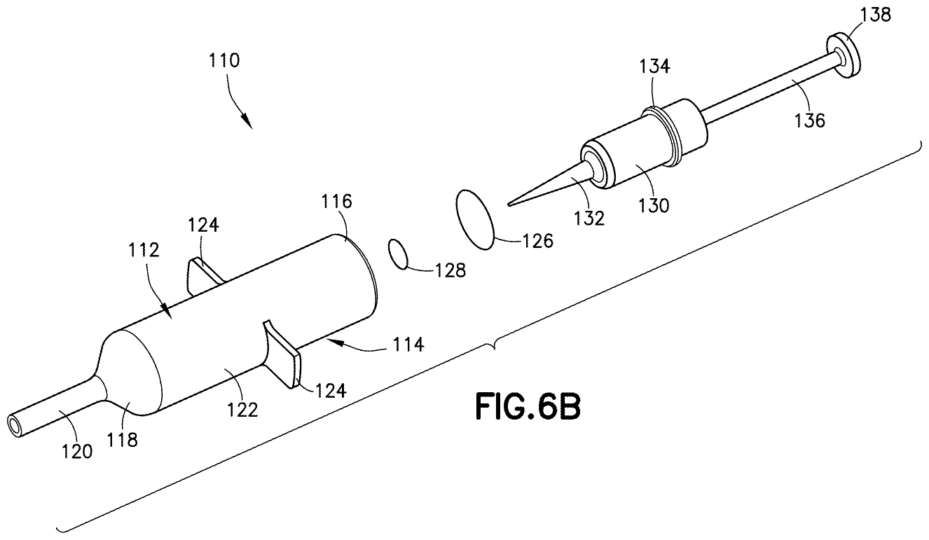

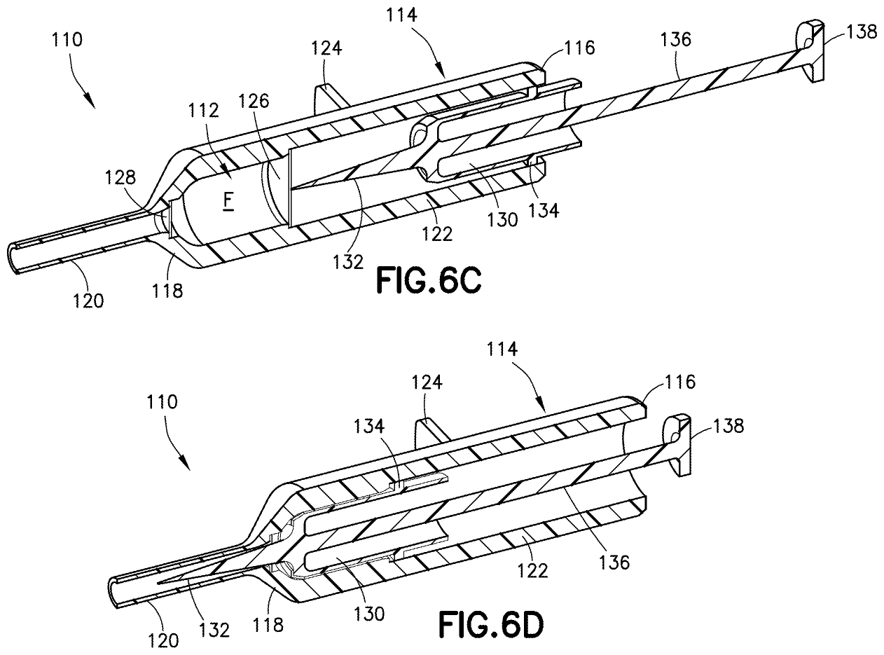

In some examples, fluid adhesive dispenser devices can include a movable stopper, plunger, or piston, similar to components used in fluid delivery syringes, which can be pressed or actuated by a user to expel the fluid adhesive F from the device. For example, with reference to FIGS. 6A-6D, an exemplary fluid adhesive dispenser device 110 includes a housing comprising a barrel 114 having a proximal end 116, a distal end 118 comprising a fluid port 120 or nozzle, and a sidewall 122 extending between the proximal end 116 and the distal end 118. The barrel 114 can be similar in shape to barrels of syringes used for fluid delivery, as are known in the art. For example, the barrel 114 can be a cylindrical structure formed from a thermoplastic material, such as polyester, polycarbonate, polypropylene, polyethylene, polyethylene terephthalate, acrylonitrile butadiene styrene, or other injection moldable or formable resin materials, as are known in the art.

In some examples, the material of the barrel can be substantially transparent so that the user can see a position of a plunger or stopper in the barrel 114 and/or how much fluid adhesive F remains in the barrel 114. Alternatively, the barrel 114 can be formed from an opaque material to, for example, protect the fluid adhesive F from sunlight or radiation, which may improve shelf life for the dispenser device 110. In some examples, the barrel 114 includes a radially extending flange or flaps 124, extending from the sidewall 122 of the barrel 114, that allow a user to hold the barrel 114 while moving a plunger or plunger rod of the dispenser device 110 through the barrel 114.

The fluid adhesive F can be sealed within a portion, chamber, or container 112 of the barrel 114 to prevent contamination of the fluid adhesive F and to ensure that the fluid adhesive F does not leak from the device 110 prior to use. For example, the dispenser device 110 can comprises a pierceable first or proximal seal 126 and a pierceable second or distal seal 128 connected to the inner surface of the sidewall 122 of the barrel 114. The proximal seal 126 can be spaced apart from the distal seal 128 defining the chamber or container 112 within the syringe barrel 114 sized to contain a single-use amount, such as less than about 1.0 mL, of the fluid adhesive F. Desirably, the space between the proximal seal 126 and the distal seal 128 is sufficient to contain a required amount of fluid adhesive F with minimal air or dead space.

The pierceable seals 126, 128 can be formed from flexible films or substrates including, for example, thermoplastic elastomer films comprising polypropylene or polyethylene, synthetic rubber, or natural rubber (e.g., isoprene). In other examples, the seals 126, 128 are formed from a metal foil, such as aluminum foil. Desirably, the seals 126, 128 are generally impervious to air and liquids, thereby protecting the fluid adhesive contained between the seals 126, 128. Specifically, the seals 126, 128 can be configured to prevent air, fluids, or other contaminants from contacting the fluid adhesive F to improve shelf life of the dispenser device 110.

The dispenser device 110 further comprises a plunger 130, which is initially positioned in the barrel 114 proximal to the proximal seal 126. The plunger 130 is configured to move through the barrel 114 to expel the fluid adhesive F from the barrel 114 through the fluid port 120 or nozzle of the barrel 114. The plunger 130 includes a piercing surface 132 configured to pierce the proximal seal 126 and/or the distal seal 128 as the plunger 130 moves through the barrel 114. The piercing surface 132 can be a sharpened or blunt point, spike, needle, or similar structure extending distally from the plunger 130 configured to pierce the seals 126, 128 as the plunger 130 moves through the barrel 114. In some examples, the piercing surface 132 is integral with other portions of the plunger 130. For example, the plunger 130 including the piercing surface 132, such as a spike, can be formed by a single molding (e.g., injection molding) process. In other examples, the piercing surface 132 (e.g., the spike) can be a separately formed structure that is attached to other portions of a plunger 130 by, for example, adhesives or ultrasonic welding. The plunger 130 can also include structures for ensuring that the plunger 130 is correctly positioned (e.g., remains substantially stable and upright) as the plunger 130 moves through the barrel 114. For example, the plunger 130 can include one or more annular ribs 134 configured to contact an inner surface of the sidewall 122 of the barrel 114 for maintaining stability of the plunger 130.

In some examples, the dispenser device 110 also includes a plunger rod 136 connected to the plunger 130 for moving the plunger 130 through the barrel 114. As with conventional plunger rods known in the art, the plunger rod 136 can include a thumb press plate 138 for pushing the plunger rod 136 distally to move the plunger 130 through the barrel 114. The plunger rod 136 can be an injection molded part formed from a rigid thermoplastic material, which can be connected to the plunger 130 by standard mechanical connectors, fasteners, or adhesives. In other examples, the plunger rod 136 can be integrally formed or co-molded with the plunger 130.

In order to use the dispenser device 110, the user first removes the device 110 (already filled with the single use amount of the fluid adhesive F) from any external packaging. When the user is ready to dispense the fluid adhesive F from the device 110, the user begins to press the plunger rod 136 through the barrel 114 by, for example, grasping the radially extending flaps 124 of the barrel 114 with his or her fingers and pressing downward (shown by arrow A7 in FIG. 6A) on the thumb press plate 138 of the plunger rod 136 with the thumb. Moving the plunger rod 136 distally through the barrel 114 brings the piercing surface 132 (e.g., spike) of the plunger 130 into contact with the proximal seal 126, as shown in FIG. 6C, causing the piercing surface 132 (e.g., spike) to pierce the proximal seal 126. Continued distal movement of the plunger 130 through the barrel 114 brings the piercing surface 132 (e.g., spike) into contact with the distal seal 128, which pierces the distal seal 128. Once the distal seal 128 is pierced, continued distal movement of the plunger 130 through the barrel 114 forces the fluid adhesive F from the barrel 114 through the fluid port 120 or nozzle of the barrel 114 to a desired site on the patient. The plunger 130 is shown in a final position, after the fluid adhesive F has been expelled from the barrel 114, in FIG. 6D. Beneficially, the user can control a rate that the fluid adhesive F is expelled from the barrel 114 based on the force applied to the plunger rod 136 and/or plunger 130.

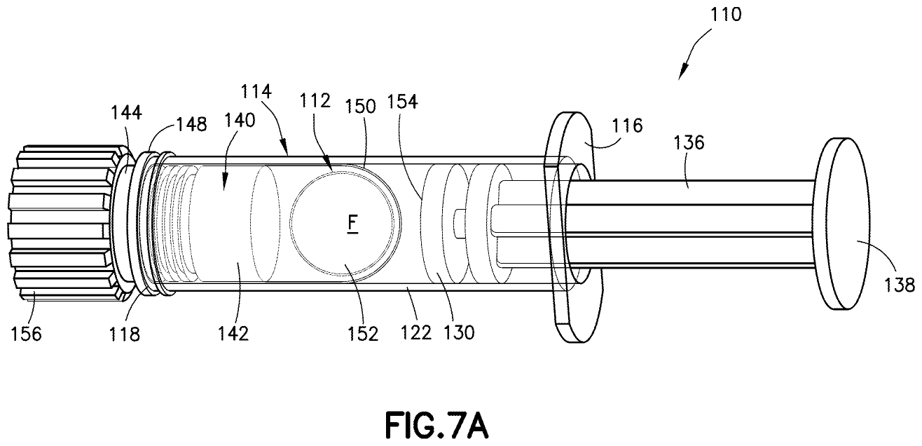

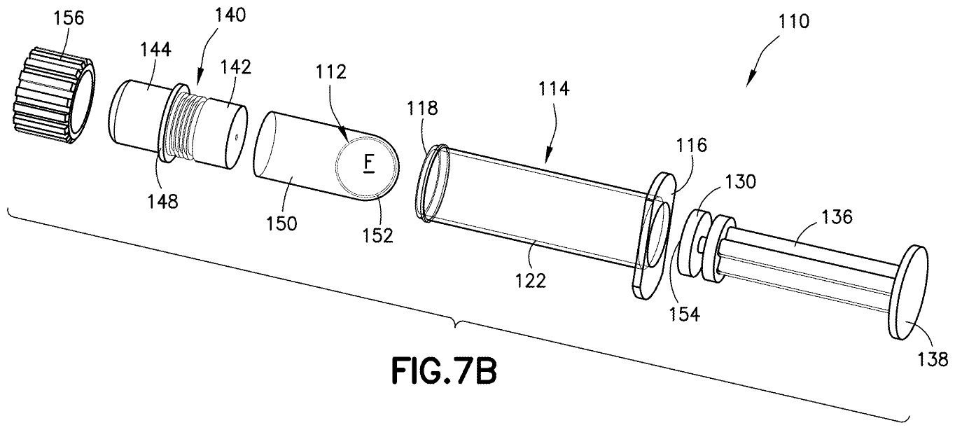

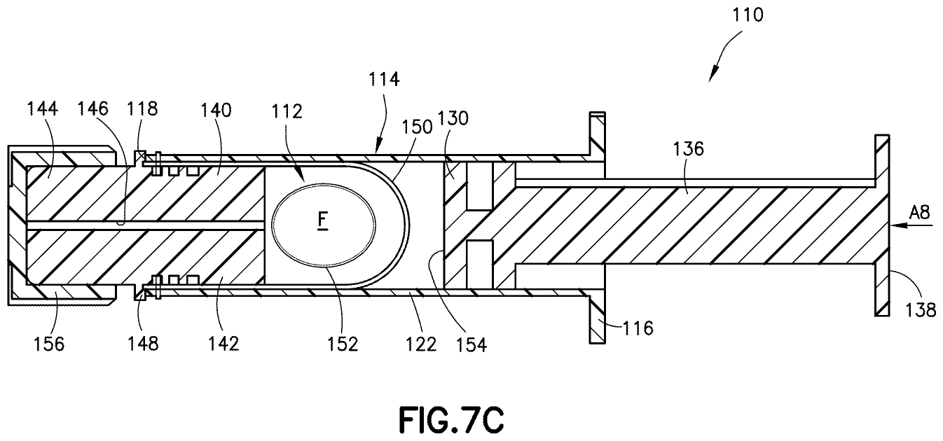

With reference to FIGS. 7A-7C, another exemplary fluid adhesive dispenser device 110 includes a barrel 114 and plunger 130 for forcing fluid adhesive F from the barrel 114 through a separate and/or removable nozzle 140. The barrel 114 can include an open proximal end 116, an open distal end 118, and a sidewall 122 extending between the proximal end 116 and the distal end 118. The device 110 also includes the tubular nozzle 140, which can be similar to the nozzle 48 of the dispenser device 10 of FIGS. 3A-3C. The tubular nozzle 140 includes a proximal portion 142 configured to be inserted in and engaged to the distal end 118 of the barrel 114 and a distal portion 144 extending from the barrel 114. The nozzle 140 also includes a lumen 146 extending through the nozzle 140 from the proximal portion 142 to the distal portion 144 thereof. The lumen 146 can be a narrow lumen sized so that the fluid adhesive F is expelled from the distal portion 144 of the nozzle 140 as drops rather than as a steady stream of fluid. The nozzle 140 can also include a radially extending flange 148 configured to be positioned against or proximate to the distal end 118 of the barrel 114 to seal the barrel 114 and to prevent the distal end portion 144 of the nozzle 140 from sliding into the barrel 114.

The dispenser device 110 can also include a dome shaped cover 150 attached to and extending proximally from the proximal portion 142 of the nozzle 140. The cover 150 can be formed from a flexible and/or compressible material. For example, the cover 150 can be formed from a metal foil or polymeric film. The cover 150 is sized to provide a cavity or space that encloses a container 112 containing the fluid adhesive F. In some examples, the proximal portion 142 of the nozzle 140 can include protrusions, such as ridges, threads, or joints configured to engage a portion of the inner surface of the cover 150 to secure the cover 150 to the nozzle 140.

As in previous examples, the dispenser device 110 also includes the container 112 enclosing the single-use amount of the fluid adhesive F. As shown in FIG. 7C, the container 112 can be a sealed bulb 152, such as a spherical bulb formed from a compressible material, such as a flexible plastic material. As shown in FIG. 7C, the bulb 152 is sized to fit between the cover 150 and the nozzle 140.

With continued reference to FIGS. 7A-7C, the dispenser device 110 further includes the plunger 130 and plunger rod 136. Unlike in previous examples, the distal surface of the plunger 130 can be a substantially flat surface 154 in order to compress the bulb 152. As in previous examples, the plunger rod 136 can be a conventional shape including, for example, a cross shaped cross-section and a thumb press plate 138. As shown in FIGS. 7A-7C, the plunger 130 is integrally formed with the plunger rod 136. For example, the plunger 130 and plunger rod 136 can be molded together by, for example, injection molding. In other examples, the plunger 130 and plunger rod 136 can be separate components connected together by an adhesive, fastener, or another connector, as are known in the art.

In some examples, the dispenser device 110 further includes a removable cap 156 attached to the distal end portion 144 of the nozzle 140. As in previous examples, the removable cap 156 is provided to prevent liquids or other debris from entering the nozzle 140, which may contaminate the device 110. The removable cap 156 also prevents the fluid adhesive F from being expelled from the dispenser device 110 until the user is ready to dispense the adhesive. As in previous examples, the removable cap 156 can be attached to the distal end portion 144 of the nozzle 140 by a threaded connection, snap fit connection, luer connection, or other connection structures, as are known in the art.

In order to use the dispenser device 110, the user first presses the plunger rod 136 in the distal direction (shown by arrow A8 in FIG. 7C) with sufficient force to rupture or burst the bulb 152, thereby releasing the fluid adhesive F from the bulb 152. When the user is ready to dispense the fluid adhesive F, the user removes the cap 156 from the distal end portion 144 of the nozzle 140. The user then continues to move the plunger 130 and/or plunger rod 136 through the barrel 114, which causes the fluid adhesive F to pass through the lumen 146 of the nozzle 140 and to be expelled from the device 110 through the distal end portion 144 of the nozzle 140.

### Fluid Adhesive Dispenser Devices with Spiked Caps and Pierceable Seals

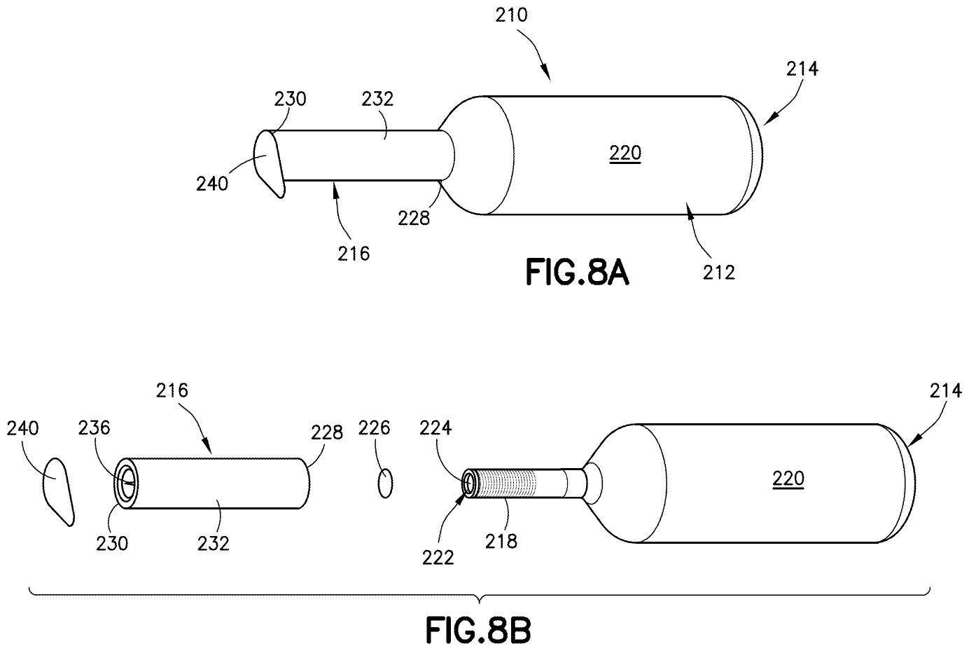

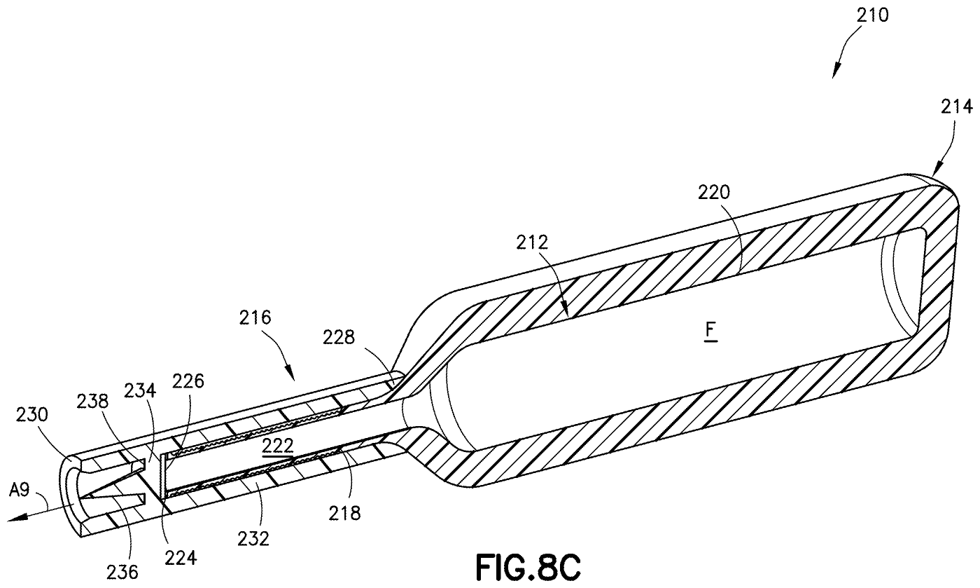

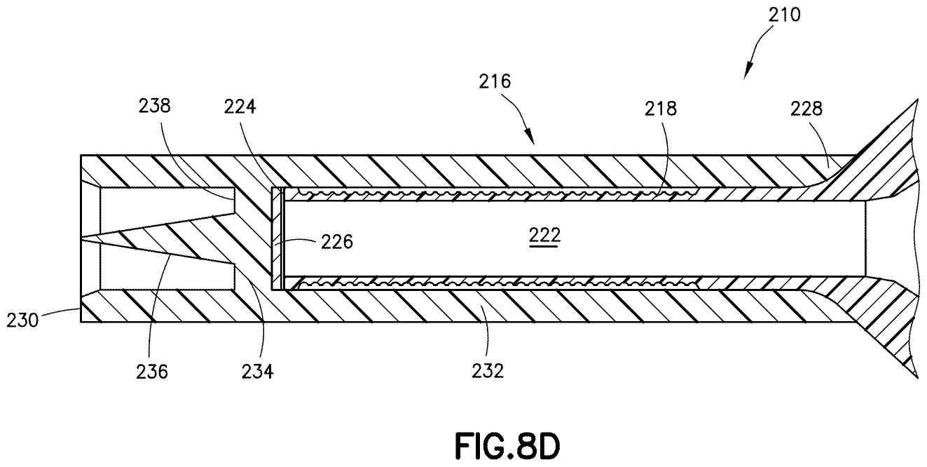

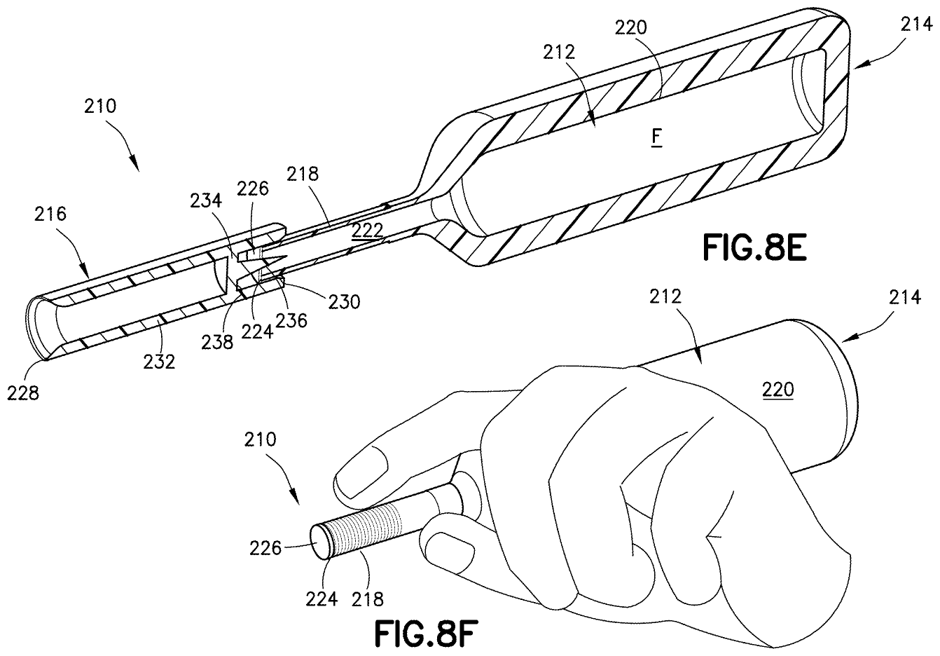

FIGS. 8A-8F show another exemplary fluid adhesive dispenser device 210 including a spiked cap 216 for piercing a sealed portion of the device 210 and an elongated tubular nozzle 218, which can be compressed and manipulated by a user to control flow of the fluid adhesive F from the device 210. The dispenser device 210 includes a housing 214 including a container 212 that defines or encloses a reservoir portion 220 configured to contain a single-use amount (e.g., less than about 1.0 mL) of the fluid adhesive F, a passageway 222 extending from the reservoir portion 220 through the elongated tubular nozzle 218, and a fluid port 224 at a distal end of the passageway 222 for expelling the fluid adhesive F from the housing 214. The housing 214 can be formed from a thermoplastic material and can include flexible areas and thicker, rigid areas that are not compressible. For example, the reservoir portion 220 and the elongated nozzle 218 of the housing 214 can be flexible and/or compressible so that a user can compress the housing 214 to control flow of the fluid adhesive F through the device 210. A central portion of the housing 214 between the reservoir portion 220 and the elongated nozzle 218 can be more rigid than the other portions of the housing 214.

The dispenser device 210 also includes a pierceable seal 226 in the passageway 222 that can be pierced to permit flow of the fluid adhesive F from the reservoir portion 220 through the passageway 222. As in previous examples, the pierceable seal 226 can be formed from a metal foil, polymer film, or metal-coated polymer film. The pierceable seal 226 can be generally impervious to liquids and air to prevent the fluid adhesive F from being expelled from the reservoir portion 220 of the housing 214 until the seal 226 is pierced and to prevent fluids and other contaminants from entering the reservoir portion 220 of the housing 214 through the passageway 222, which could contaminate the device 210.

The dispenser device 210 further comprises the spiked cap 216 attached to the elongated nozzle 218 for preventing the fluid adhesive F from being expelled through the port 224 until the user is ready to dispense the fluid adhesive F from the device 210. As shown in FIG. 8C, the cap 216 comprises an open proximal end 228 configured to slide over the port 224 and nozzle 218 of the housing 214, an open distal end 230 configured to extend away from the housing 214, and a cylindrical sidewall 232 defining a cylindrical channel extending between the proximal end 228 and the distal end 230 of the cap 216. The cap 216 also includes a wall 234 extending through the cylindrical channel of the cap 216. The cap 216 further includes a pointed surface or spike 236 extending from a distal surface 238 of the wall 234 towards the distal end 230 of the cap 216. The pointed surface or spike 236 is configured to pierce the seal 226 when the spike 236 is inserted into the passageway 222 of the housing 214. The cap 216 can also include a removable packaging cover 240 over the distal end 230 of the cap 216 that covers the spike 236 prior to use of the device 210. For example, the packaging cover 240 can be secured to the distal end 230 of the cap 216 by a sticker or adhesive. The removable packaging cover 240 can be removed from the cap 216 when the device 210 is ready for use.

As shown in FIGS. 8A, 8C, and 8D, the dispenser device 210 is initially provided in a first orientation with the spike 236 pointing away from the port 224 of the housing 214. In order to pierce the seal 226 and prepare the device 210 for use, the cap 216 can be removed from the port 224 or nozzle 218 and reconnected to the port 224 or nozzle 218 in a second orientation with the spike 236 inserted into the passageway 222 through the port 224 to pierce the seal 226.

As previously described, in some examples, the elongated nozzle 218 is a compressible tubular portion configured to be compressed to control flow of the fluid adhesive F through the port 224 of the housing 214. For example, as shown in FIG. 8F, a user can grasp the dispenser device 210 like a pen, with the index finger and thumb contacting the elongated nozzle 218 of the dispenser device 210. When the user applies substantial force to the elongated nozzle 218, fluid flow from the fluid reservoir 220 through the device 210 is prevented. When the user applies less force to the elongated nozzle 218, fluid flow through the passageway 222 and nozzle 218 is permitted so that the fluid adhesive F can be expelled from the dispenser device 210.

In some examples, the removable cap 216 is configured to cover or surround the elongated nozzle 218 of the device 210. For example, as shown in FIG. 8D, the proximal end portion 228 of the cap 216 can surround the elongated nozzle 218 with the port 224 or distal end 230 of the elongated nozzle 218 positioned adjacent to the wall 234 of the cap 216.

In order to use the device 210, a user first removes the packaging cover 240 from the distal end 230 of the cap 216 to expose the spike 236. The user then removes the cap 216 from the port 224 or elongated nozzle 218 by pulling the cap 216 away from the port 224 and elongated nozzle 218 in a direction shown by arrow A9 (in FIG. 8C). After the user removes the cap 216, the user inverts the cap 216 and then pushes the cap 216 onto the port 224 and nozzle 218 causing the spike 236 to be inserted into the elongated nozzle 218, as shown in FIG. 8E. Pushing the spike 236 into the nozzle 218 causes the spike 236 to contact and pierce the seal 226, thereby permitting the fluid adhesive F to flow into the passageway 222 of the nozzle 218. When the user is ready to dispense the fluid adhesive F from the device 210, the user removes the cap 216 from the port 224 or nozzle 218 by pulling the cap 216 away from the port 224 and nozzle 218. The user then holds the device 210 with the elongated nozzle 218 between the index finger and thumb (as shown in FIG. 8F) to control flow of the fluid adhesive F through the passageway 222 of the nozzle 218. The user can also compress the fluid reservoir portion 220 of the housing 214 to force the fluid adhesive F from the fluid reservoir 220 into the passageway 222. In some examples, holding the dispenser device 210 in a vertical or upright orientation may cause the fluid adhesive F to drain from the fluid reservoir 220 more quickly due to gravity than if the device 210 is held in other orientations.

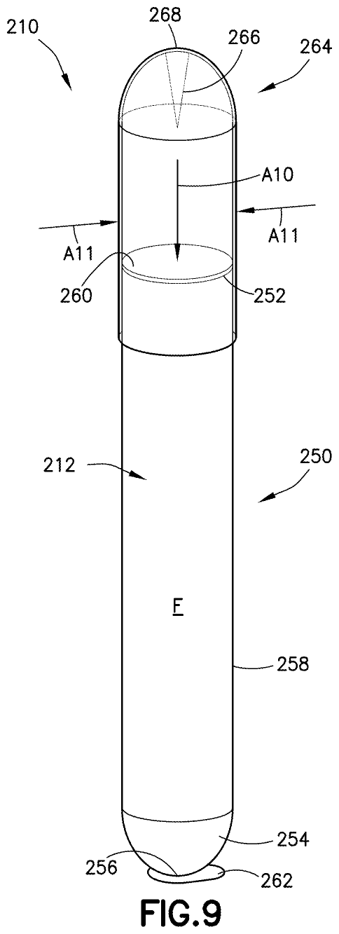

With reference to FIG. 9, another exemplary fluid adhesive dispenser device 210 includes a housing 214 comprising a barrel 250 having an open proximal end 252, an open distal end 254 comprising a port 256 for expelling a fluid adhesive F from the barrel 250, and a sidewall 258 extending between the proximal end 252 and the distal end 254. The dispenser device 210 further comprises a proximal seal 260 over the open proximal end 252 of the barrel 250. As in previous examples, the proximal seal 260 can be formed from a pierceable, fluid impervious material in order to protect and/or prevent contamination of a single-use amount of a fluid adhesive F contained within the barrel 250. The device 210 can also include a distal seal or packaging cover 262 over the open distal end 254 of the barrel 250. As in previous examples, the distal seal or packaging cover 262 can be a sticker adhered to the distal end 254 of the barrel 250, which can be removed by grasping the packaging cover 262 and pulling it away from the barrel 250. The packaging cover 262 is configured to be removed from the barrel 250 prior to use so that the fluid adhesive F can be expelled from the barrel 250. The proximal seal 260 is configured to be pierced by a spike or needle. The proximal seal 260 and packaging cover 262 are configured to seal the fluid adhesive F within an interior, chamber, or container 212 of the barrel 250 until a user is ready to dispense the fluid adhesive F from the dispenser device 210.

As shown in FIG. 9, the dispenser device 210 further comprises a cap 264 or rubber dropper affixed to the proximal end 252 of the barrel 250. The cap 264 can be a dome-shaped structure formed from a flexible, bendable, and/or compressible material having an opening sized to be positioned over the proximal end 252 of the barrel 250. The cap 264 includes a spike 266 or pointed needle extending from an inner surface of the cap 264. The cap 264 is configured to be compressed by, for example, applying sufficient force to a proximal end 268 of the cap 264, which causes the spike 266 or pointed needle to move in a direction (shown by arrow A10 in FIG. 9) toward the proximal seal 260 to pierce the proximal seal 260. Once the proximal seal 260 is pierced, a user can repeatedly compress and release the cap 264, as shown by arrows A11 (in FIG. 9), in order to pump or push the fluid adhesive F through the barrel 250, thereby expelling the fluid adhesive F through the port 256 at the distal end 254 of the barrel 250.

In order to use the dispenser device 210, the user first presses downward on the proximal end 268 of the cap 264, causing the spike 266 to pierce the proximal seal 260. When the user is ready to dispense the fluid adhesive F from the dispenser device 210, the user removes the distal seal or packaging cover 262 from the distal end 254 of the barrel 250. Once the proximal seal 260 is pierced and the packaging cover 262 removed, the user repeatedly compresses and releases the cap 264, which pushes bursts of air through the barrel 250 causing the fluid adhesive F to move through the barrel 250 towards the open distal end 254 and port 256 of the barrel 250. The fluid adhesive F is expelled from the barrel 250 through the port 256. Depending upon a size and shape of the barrel 250 and/or the port 256, the fluid adhesive F can be expelled from the barrel 250 as a steady stream or as individual droplets. The user continues to pump the cap 264 by repeatedly compressing and releasing the cap 264 until a desired amount of the fluid adhesive For all of the fluid adhesive F in the barrel 250 is expelled from the device 210.

### Manufacturing Methods for Fluid Adhesive Dispenser Devices

As previously described, the fluid adhesive dispenser devices 10, 110, 210 of the present disclosure are configured to be provided as pre-filled devices that contain the single-use amount of the fluid adhesive. Advantageously, the devices 10, 110, 210 are configured so that the entire filled device can be sterilized during a single terminal sterilization process. By contrast, many conventional dispenser devices are sterilized in separate stages. For example, a conventional device may require that an ampule or container containing a medical fluid is sterilized separately from other components or parts of the device. Only after the different parts are separately sterilized can the device be assembled and provided to a user. By allowing the entire filled device to be sterilized in a final terminal process, the fluid adhesive dispenser devices 10, 110, 210 of the present disclosure can be manufactured and prepared for use in an efficient manner that saves time compared to other dispenser devices.

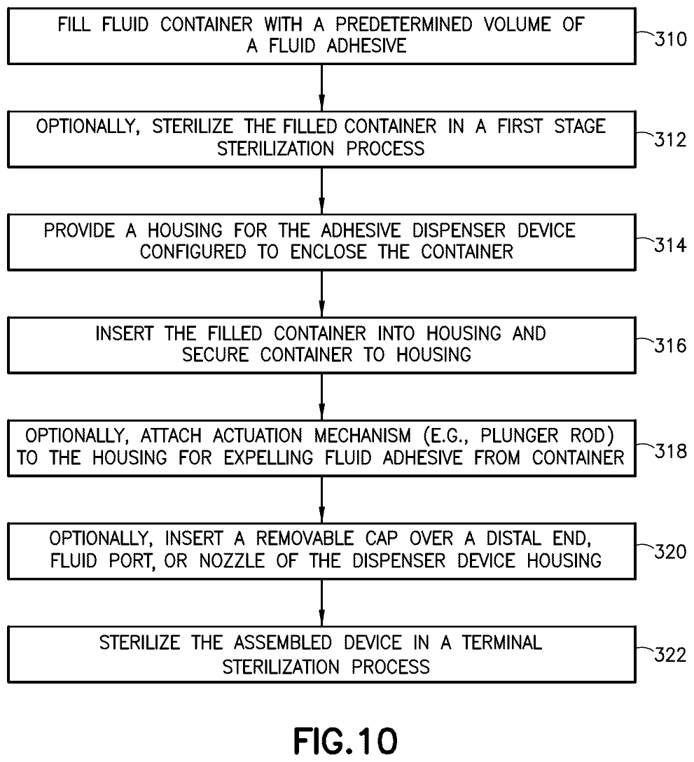

FIG. 10 is a flow chart showing steps of a manufacturing method for producing the fluid adhesive dispenser devices 10, 110, 210 of the present disclosure. At step 310, the method includes filling a container 12, 112, 212, such as an ampule, bulb, or syringe barrel, with a predetermined volume of the fluid adhesive. After filling, at step 312, the method optionally includes sterilizing the container 12, 112, 212 by a conventional sterilization process used for medical devices, as is known in the art, such as by exposing the filled container 12, 112, 212 to radiation (e.g., ultraviolet light, heat, or infrared radiation) or to a chemical gas (e.g., ethylene oxide, vaporized peracetic acid, nitrogen dioxide, etc.).

At step 314, the method further includes providing a housing of the dispenser device 10, 110, 210, such as a compressible bellows, syringe barrel, or foil pouch. At step 316, the method can further include inserting the filled container 12, 112, 212 into an appropriate portion of the housing. For example, as previously described, the filled container 12, 112, 212 can be secured in the housing by heat sealing, adhesives, or mechanical connectors or fasteners. At step 318, optionally, the method further includes attaching an actuation structure, such as a plunger and/or plunger rod, to the housing so that the fluid adhesive can be manually expelled from the device 10, 110, 210. For example, as previously described, the plunger and plunger rod can be used to burst or rupture the filled container 12, 112, 212 and to move the fluid adhesive toward a distal end or fluid port of the device 10, 110, 210. At step 320, the method can further include inserting a removable cap over a distal end, fluid port, or nozzle of the device to prevent contamination of the device and to ensure that the fluid adhesive is not released from the device 10, 110, 210 at unexpected times.

At step 322, after the device 10, 110, 210 is fully assembled with the filled container 12. 112, 212 in the device housing, the method further includes sterilizing the entire device 10, 110, 210 including the housing and filled container 12, 112, 212 in a terminal sterilization process. For example, as previously described, the sterilization process can include exposing the entire device 10, 110, 210 to radiation or a chemical gas. Desirably, the sterilization process is selected so that the fluid adhesive in the filled container 12, 112, 212 remains substantially unpolymerized for at least 24 months following sterilization.

While examples of the fluid adhesive dispenser devices and methods of the present disclosure are shown in the accompanying figures and described hereinabove in detail, other examples will be apparent to, and readily made by, those skilled in the art without departing from the scope and spirit of the invention. Accordingly, the foregoing description is intended to be illustrative rather than restrictive. The invention described hereinabove is defined by the appended claims and all changes to the invention that fall within the meaning and the range of equivalency of the claims are to be embraced within their scope.

1. A fluid adhesive dispenser device, comprising:
a housing comprising a passageway through the housing and a port at a distal end of the passageway; and
a container at least partially within the housing configured to contain a single-use amount of the fluid adhesive,
wherein the container is configured to be opened allowing the fluid adhesive contained therein to be actively expelled from the container through the passageway and the port of the housing.

2. The device of claim 1, wherein the fluid adhesive is expelled from the housing through the port of the housing in a direction-independent manner.

3. The device of claim 1, wherein the single-use amount of the fluid adhesive is less than about 1.0 mL.

4. The device of claim 1, wherein the fluid adhesive is actively expelled from the container by reducing a fluid-containing volume of the container, thereby forcing fluid from the container into the passageway extending through the housing.

5. The device of claim 1, wherein the fluid adhesive comprises a tissue adhesive configured to be used for securing a vascular access device to skin of a patient, wound closure, and/or insertion site closure.

6. The device of claim 1, wherein the fluid adhesive comprises a cyanoacrylate composition.

7. The device of claim 1, wherein the container is configured to be opened by at least one of compressing the container to create one or more openings in the container, rupturing the container, breaking the container, or piercing a portion of the container.

8. The device of claim 1, wherein the housing comprises a compressible bellows configured to be compressed by a user to compress the container in order to expel the fluid adhesive from the container.

9. The device of claim 1, wherein the housing comprises a pouch comprising a metal-coated polymer film defining cavity sized to receive the container, and wherein the container comprises a sealed ampule or bulb positioned in the cavity of the pouch.

10. The device of claim 1, wherein the port comprises an elongated tubular nozzle configured to be manually compressed by a user to control fluid flow through the nozzle.

11. The device of claim 1, wherein the housing comprising at least one tab configured to contact an outer surface of the container to rupture the container,
wherein applying force to the at least one tab of the housing in a first direction ruptures the container establishing fluid communication between an interior of the container and the passageway of the housing, and
wherein applying force to the housing in a second direction moves the container towards the port of the housing, thereby expelling the fluid adhesive from the interior of the container through the passageway and the port of the housing.

12. The device of claim 1, wherein the container comprises at least one pierceable seal that can be pierced to establish fluid communication between an interior of the container and the passageway of the housing.

13. The device of claim 1, further comprising a filter within the passageway of the housing configured to separate broken pieces of the container from the fluid adhesive prior to expulsion of the fluid adhesive from the port of the housing.

14. The device of claim 1, wherein the housing comprises a barrel comprising a proximal end, a distal end comprising the port, and a sidewall extending between the proximal end and the distal end,
the device further comprising a plunger configured to move through the barrel to expel the fluid adhesive from the container through the passageway of the housing.

15. The device of claim 14, wherein the container comprises proximal and distal pierceable seals connected to an inner surface of the sidewall of the barrel, and wherein the fluid adhesive is contained between the first and second seals.

16. The device of claim 15, wherein the plunger comprises a piercing surface configured to pierce the proximal and/or distal seals, and wherein advancing the plunger through the barrel pierces the proximal seal and forces the fluid adhesive through the barrel and through the port.

17. The device of claim 1, further comprising a removable cap attached to the port for preventing the fluid adhesive from being expelled through the port until the cap is removed.

18. The device of claim 17, wherein the container comprises a pierceable seal for retaining the fluid adhesive in the container,
wherein the cap comprises a spike configured to pierce the seal when the spike is inserted into the passageway of the housing,
wherein the cap is initially provided in a first orientation with the spike pointing away from the port of the housing, and
wherein the cap is configured to be removed from the port and reconnected to the port in a second orientation with the spike inserted into the port to pierce the seal.

19. A method of manufacture of a fluid adhesive dispenser, the method comprising:
providing a fluid adhesive dispenser device, comprising:
a housing comprising a passageway through the housing and a port at a distal end of the passageway; and
a container at least partially within the housing and filled with a single-use amount of the fluid adhesive and,
wherein the container is configured to be opened allowing the fluid adhesive contained therein to be actively expelled from the container through the passageway and the port of the housing; and
sterilizing the filled dispenser device.

20. A fluid adhesive dispenser device, comprising:
a housing comprising a passageway through the housing and a port at a distal end of the passageway;
an ampule at least partially within the housing configured to contain a single-use amount of the fluid adhesive and configured to be ruptured so that the fluid adhesive passes from an interior of the ampule to the passageway of the housing; and
a filter within the passageway of the housing configured to separate broken pieces of the ruptured ampule from the fluid adhesive prior to expulsion of the fluid adhesive from the port of the housing.
